(19) United States
(12) Patent Application Publication
(10) Pub. No.: US 2017/0028149 A1
(43) Pub. Date: Feb. 2, 2017

(54) POSITIONING DEVICE AND METHOD FOR USE WITH A PATIENT UNDER ANESTHESIA

(71) Applicant: REVOLUTIONARY MEDICAL DEVICES, INC., Tucson, AZ (US)

(72) Inventors: Michael J. Pedro, Brooklyn, NY (US); Steven H. Cataldo, New York, NY (US); James Ellis, Tempe, AZ (US); John Hunt, Tucson, AZ (US); David M. Kane, Tucson, AZ (US); Thomas Reilly, Tucson, AZ (US)

(21) Appl. No.: 15/217,753

(22) Filed: Jul. 22, 2016

Related U.S. Application Data
(63) Continuation of application No. 14/901,647, filed on Dec. 28, 2015.
(60) Provisional application No. 61/840,980, filed on Jun. 28, 2013.
(60) Provisional application No. 61/840,997, filed on Jun. 28, 2013.
(60) Provisional application No. 61/876,093, filed on Sep. 10, 2013.
(60) Provisional application No. 61/907,938, filed on Nov. 22, 2013.
(60) Provisional application No. 61/910,648, filed on Dec. 2, 2013.
(60) Provisional application No. 61/924,114, filed on Jan. 6, 2014.
(60) Provisional application No. 61/925,089, filed on Jan. 8, 2014.
(60) Provisional application No. 61/941,206, filed on Feb. 18, 2014.
(60) Provisional application No. 61/979,912, filed on Apr. 15, 2014.
(60) Provisional application No. 61/983,941, filed on Apr. 24, 2014.
(60) Provisional application No. 62/007,802, filed on Jun. 4, 2014.

(51) Int. Cl. A61M 16/06; A61M 16/01; A61G 13/12; A61M 16/20; A61M 16/10; A61M 16/00; A61M 16/08

(52) U.S. Cl. CPC A61M 16/0622; A61M 16/0078; A61M 16/01; A61M 16/0875; A61M 16/085; A61M 16/208; A61M 16/10; A61M 16/009; A61M 16/0683; A61G 13/121; A61G 13/122; A61M 2202/0208

(57) ABSTRACT

An apparatus for supporting the head and neck for airway management and to facilitate the maintenance of a patent airway under anesthesia, for unconscious patients, and for any circumstance requiring a patent airway while the patient is lying on her side. The apparatus includes a head supporting surface, an adjustable neck supporting surface, and two adjustable jaw support arms to protrude the jaw forward and maintain ventilation while the patient is lying on either of his/her sides.

### CROSS REFERENCE TO RELATED APPLICATIONS

This application is a continuation of U.S. application Ser. No. 14/901,647, filed Dec. 28, 2015, which in turn claims priority from PCT Patent Application Serial No. PCT/US14/044934, filed Jun. 30, 2014, which claims priority from U.S. Provisional Application Ser. No. 61/840,980, filed Jun. 28, 2013, and from U.S. Provisional Application Ser. No. 61/840,997, filed Jun. 28, 2013, and from U.S. Provisional Application Ser. No. 61/876,093, filed Sep. 10, 2013, and from U.S. Provisional Application Ser. No. 61/907,938, filed Nov. 22, 2013, and from U.S. Provisional Application Ser. No. 61/910,648, filed Dec. 2, 2013, and from U.S. Provisional Application Ser. No. 61/924,114, filed Jan. 6, 2014, and from U.S. Provisional Application Ser. No. 61/925,089, filed Jan. 8, 2014, and from U.S. Provisional Application Ser. No. 61/941,206, filed Feb. 18, 2014, and from U.S. Provisional Application Ser. No. 61/979,912, filed Apr. 15, 2014, and from U.S. Provisional Application Ser. No. 61/983,941, filed Apr. 24, 2014, and from U.S. Provisional Application Ser. No. 62/007,802, filed Jun. 4, 2014, the contents of which are incorporated herein by reference.

### BACKGROUND OF THE INVENTION

The present invention in one aspect relates to a positioning device and method to facilitate the maintenance of a patent airway while a patient is either unconscious, unable to maintain a patent airway, or under sedation and/or anesthesia, which causes an airway to collapse. In another aspect, the present invention relates to a positioning device to facilitate the maintenance of a patent airway while a patient is either unconscious, unable to maintain a patent airway, or under sedation and/or anesthesia by accurately positioning the patient's head, neck, torso, and jaw and to enable the most optimal view if endotracheal intubation is required by aligning three axes (oropharyngeal, laryngeal, tracheal) prior to placing an endotracheal tube, simultaneously while trying to place an endotracheal tube, and during extubation. In yet another aspect the present invention relates to an anesthesia nasal mask, full face mask, and combination nasal-oral mask, which can be used for oxygenation and ventilation both prior to intubation, during intubation, and after intubation, can be connected to either a portable oxygen supply source and used to supply oxygen for transport and/or connected to an End-tidal CO2 monitor to measure CO2 levels, and/or to help maintain patient positioning. In still yet another aspect, the invention relates to a device for facilitating jaw thrust of a patient either unconscious, unable to maintain a patent airway, or undergoing anesthesia.

During surgery a patient is usually placed under anesthesia and the most common delivery system consists of canisters containing anesthesia gases and oxygen, a system of regulating the gas flow and the patient's breathing, and a device ensuring the potency of the patient's airway for breathing/ventilation, oxygenation and the delivery of the anesthetic gas mixture. Currently, a full face mask is used to provide oxygen to the patient either before the patient is anesthetized, and to supply oxygen, remove carbon dioxide (CO2), and supply anesthetic gases while the patient is anesthetized. A few of the drawbacks of current full face mask ventilation is that it first requires constant contact between the provider's hands and the patient's face to hold the mask in place and keep the patient in the so-called sniffing position in order to ensure that oxygen and anesthetic gases do not leak out into the air and that the patient's airway remains patent. If the provider fails to maintain the patient in the sniffing position, a dangerous complication known as upper airway obstruction may occur where the soft palate and/or tongue collapse into the airway. The reason the provider needs to perform continuous mask holding and maneuvering is the human anatomy and physiology. When muscles of the jaw, soft palate, tongue, and upper airway relax due to obstructive sleep apnea, sedatives and/or muscle relaxants given to the patient for sedation and/or anesthesia, the upper airway (mouth, pharynx, larynx) may become partially obstructed and possibly completely closed. When either the head of the patient falls forward or the jaw drops back, either the tongue and/or the soft palate falls back into the airway resulting in snoring (partial obstruction) or apnea (complete inability for oxygen to pass via the upper airway into the lungs). Should this occur, the patient's head and neck should be properly positioned and either non-invasive positive pressure ventilation such as continuous positive airway pressure (CPAP) ventilation or Bilevel positive airway pressure ventilation BiPAP) and/or a so-called “jaw thrust” maneuver” should be attempted, as will be discussed below. A second drawback of the current full facemask is that a provider must remove the mask prior to intubation, since the mask covers the patient's mouth and prevents a laryngoscope from entering it. Also, current nasal masks have the anesthesia circuit coming from the right side of the patient and connecting to the nasal mask aperture in the middle of the nasal mask. Since the anesthesia circuit comes from the right side it blocks the provider from being able to intubate, because all intubations are performed on the right side. Also, since the anesthesia circuit connects to the middle aperture of the nasal mask, both the nasal mask connection and the anesthesia circuit obstruct the view of the patient's mouth if a provider was to attempt to intubate a patient. Therefore both the current nasal mask and the full face mask must be removed prior to an intubation attempt is made, the provider can therefore no longer try to oxygenate or ventilate a patient until successful placement of an endotracheal tube occurs. This is also known as the apneic period and one of the most critical events in airway management. The present invention will only cover the patient's nose when attempting direct laryngoscopy and placement of an endotracheal tube, allowing the provider to continue oxygenating and ventilating the patient, and will not obstruct the view of the provider while he/she performs direct laryngoscopy, as the anesthesia circuit connector aperture within the proposed nasal and oral-nasal mask will be located on either the left or right side of the nasal and oral-nasal mask (not sticking straight up) and allow the anesthesia circuit to also be on either the left or right side of the patient, which is out of the way of the provider's view when performing direct laryngoscopy. The present invention essentially eliminates the critical apneic period. A third drawback of the current full facemask is it cannot be used as a source of oxygen for patients during transport unless it is connected to a resuscitator bag. Currently, at the end of each case, anesthesiologists throw the anesthesia mask away since it cannot be used to transport patients to the Post-Anesthesia Care Unit (PACU). The reason why the current nasal mask or full face mask cannot be used for transport is because they only have one aperture which must connect to a non-invasive positive pressure device which has both inspiratory and expiratory valves. These masks do not have a vent to allow for inhalation and exhalation when attached to oxygen supply devices. The present invention has an additional port where either oxygen from an oxygen supply device can attach or an end-tidal CO2 monitor can attach and the anesthesia circuit aperture can either be used as a vent to prevent excessive pressure from being built up, connected to a resuscitator bag and be used for bag-mask ventilation, or connected to a non-invasive positive pressure ventilation device (CPAP, BiPAP, etc) to assist in ventilation. The present invention's one or more extra port/s (oxygen/CO2) can also be covered and the anesthesia circuit can be connected to the anesthesia circuit connector aperture to be used for non-invasive positive pressure ventilation. The present invention's one or more extra port/s (oxygen/CO2) can also be attached to either an oxygen monitor and/or an end-tidal CO2 monitor and the anesthesia circuit can be connected to the anesthesia circuit connector aperture to be used for non-invasive positive pressure ventilation while being able to measure oxygen levels and CO2 levels.

Another problem exists when a provider fails to administer enough anesthesia or sedation and it begins to wear off and the patient begins to move. This can also cause the patient's airway to obstruct as well since the patient's head and neck position are no longer in the sniffing position. Patient movement during surgery also can be dangerous because it can cause the surgeon to make a mistake, particularly in eye, ear, nose, neck, head, and throat surgery.

Also, over the last decade the number of Monitored Anesthesia Care (MAC) cases, especially colonoscopies, have dramatically increased (several million being performed annually just in the U.S.), and unfortunately, so have airway complications resulting in both death and brain damage. MAC cases use sedating medications in order to limit the amount of physical and psychological pain that the patient may experience. However, these sedating medications can cause relaxation of the muscles that help maintain an open airway. Relaxation of these muscles can then lead to the airway becoming obstructed (i.e., upper airway obstruction) and stopping the patient from breathing. Also, if a higher than expected dose of sedating medication is given it can lead to respiratory depression where the patient's brain fails to communicate when to take a breath.

Historically, in order to reduce the risk of an upper airway obstruction, a provider would first change the position of the patient's head by lifting the chin or by having the patient lie on their side. Lifting the chin places the patient in the so-called “sniff position” or “sniffing position” and allows the mandible to be slightly displaced anteriorly which removes the tongue from the airway. See Cattano et al, Airway Management and Patient Positioning: A Clinical Perspective, Anesthesiology News Guide to Airway Management, P. 15 (2011). The sniffing position also aligns three axes (oropharyngeal, laryngeal, tracheal) and gives the provider the most optimal view for intubation. Laying a patient on his or her side prevents gravity from forcing the patient's tongue and/or soft palate into the patient's airway and blocking it by allowing the tongue and soft palate to extend forward. However, these two positions only prevent upper airway obstruction in about half of the patients. A new technique that can be used based off of current evidence is to apply nasal CPAP in patients with upper airway obstruction as it is more effective than full face mask CPAP. If these maneuvers fail to relieve the upper airway obstruction, the provider then will perform a jaw thrust maneuver. The jaw thrust maneuver is done with one hand moving the jaw up and forward to move the tongue so that the airway is opened. The jaw thrust is performed while holding a mask over the patient's mouth and nose to deliver oxygen. In order to ventilate the patient while performing a jaw thrust maneuver, the provider is required to hold the mask over the patient's face almost constantly and prevents the ability to perform other tasks during the surgery. An obvious disadvantage of this maneuver is the use of two hands. This can become especially cumbersome when the patient is in the lateral position because the side of the jaw that the patient is lying on cannot be reached.

In a 2000 study, 11% of operations for patients utilizing a full face mask and jaw thrust for airway management had sore-jaw complaints. Currently, 7% of anesthesiologists have lawsuits associated with complications related to anesthesia and jaw-thrust related trauma. Additionally, the requirement to multi-task (perform jaw thrust and other duties) simultaneously results in a significant opportunity for error. This translates into poor patient outcomes and liability for both the anesthesiologist and the facility. This has led to a significant loss of popularity of the mask anesthetics and the increased use of other airway devices, which are more invasive and have greater potential side effects and complications. A successful hands-free jaw thrust device program would benefit more than 13,000,000 patients undergoing anesthesia in the Unites States annually, as well as the doctors and facilities providing the service.

Another difficulty that is encountered with the use of a full face mask is maintaining the full face mask on the face of the patient during the procedure, and especially during a long cases since not all faces are the same size or shape and face masks are manufactured in only a few different sizes. Also, patient movement can cause a face mask to fall off, as can incidental contact. In order to maintain a face mask on a patient, the current procedure is to employ a ring and strap member configuration. As best shown in Helling's, U.S. Pat. No. 5,975,079, the ring member typically includes a ring having a central aperture that is sized to interiorly receive a generally cylindrical gas port connector, so that the ring can fit over the cylindrical gas port connector. The ring includes a plurality (usually 4 or 6) radially extending arms that are spaced in intervals around the circumference of a cylindrical part of the ring. An upwardly extending prong is formed on the distal (radially outward most) portion of each of the radially extending arms, and serves as a prong or stud member. This approach have several disadvantages. The first disadvantage is that the prongs are sharp and have been known to cause abrasions to both the provider and the patient. Another disadvantage is that the head strap must be placed beneath the patient's head and the extensions then fixed to the prongs in front. This becomes challenging if the patient has already placed his or her head down on the surface, the provider now has to lift the patient's head up. Also, some patients have difficulty flexing their neck. Additionally, if the patient has long hair, the strap may become tangled in the patient's hair. Another disadvantage is that the strap is bulky, consisting of four long extensions and a very wide head strap. The size and bulkiness of the strap has the potential to create additional clutter around the patient's face, cause corneal abrasions, and may impair a surgeons' or nurses' ability to work on the patient. Yet another disadvantage of the Helling device is that it cannot fix the patient's head to the surface. Thus, even though it is purported to be a hands free approach, since the patient's head is not fixed to the surface, movement of the patient's head or neck can cause obstruction of the patient's airway and prevent oxygen and/or anesthetic gases from being delivered to the patient. Finally, the Helling device is very cumbersome to be used for transport since it tightly covers the nose and mouth causing the patient to feel like they are suffocating and unless they are transported with a resuscitator bag, the current full face mask cannot be connect to an oxygen supply device, since the mask does not contain a vent and or inspiratory/expiratory valves. Other patents which teach systems for stabilizing face masks or binding a person to a stretcher or the like include U.S. Pat. Nos. 6,981,503; 7,753,051; 4,905,712; 3,889,668; 3,897,777, and published Applications Nos. 2009/0178682 and 2007/0295335.

Another particular and growing problem involves difficulties in ventilation and intubation of obese patients, which problem is becoming more prevalent as the general population is becoming more obese. Obese patients not only have more soft tissue in their upper airways that leads to obstruction, but they also have a significant amount of extra weight that compresses their chest, restricting air exchange. Historically, a health care provider would place sheets or blankets under the patient's back, shoulders, neck, and head in order to allow gravity to relieve the extra weight. This has been shown to be only slightly effective compared to a more rigid structure like a ramp, which has shown to be very effective. Also, if an endotracheal tube is not in place at the start of the case because the patient is only undergoing sedation and during the case the patient goes into respiratory failure and requires endotracheal intubation, there is not enough time to place these sheets under the patient and ensure proper positioning. Moreover, it requires health care personnel to lift these morbidly obese patients in order to position the sheets or blankets underneath the obese patients and to remove the sheets or blankets once the surgery starts. Obviously this greatly increases the risk of work related injuries. Since obese patients are also known to be more difficult intubations, a ramp, in combination with the “sniffing position” and the “ear aligned with sternum position” has been proven to more effective than “sniffing position alone”. Although the medical literature specifies the most precise angles for the “sniffing position” which aligns three axes (oropharyngeal, laryngeal, and tracheal) and gives a health care provider the most optimal view for intubation as well as the most optimal patent airway, there currently are no devices that can either assure the provider that the patient's head and neck angles are properly aligned or changed the glottic view in real time during intubation. Accordingly, the health care provider has to “eye ball” these angles which obviously results in errors.

The prior art has proposed various devices for facilitating maintenance of a patent airway. For example, U.S. Pat. Publication No. 2012/0180220 shows an apparatus for supporting the head and neck of a user for airway management includes a head-supporting surface dimensioned to receive and support the head of the user and a neck-supporting surface connected to the head-supporting surface, wherein the neck-supporting surface is dimensioned to receive and support the neck of the user; wherein the head-supporting surface and neck-supporting surface are configured so that when the user is lying on his or her side with a side of his or her head positioned on the head-supporting surface and a side of his or her neck on the neck-supporting surface, the user's head and neck are automatically aligned in the sniff position for improved airway management. However, this patent lacks the abilities to independently adjust either the head or the neck as well as adjust them during the most critical point, intubation. It is also unable to custom fit head, neck, and torso positioning or perform a jaw thrust, which is critical in being able to maintain a patent airway. This device also cannot restrain a patient's head during patient movement. U.S. Pat. No. 8,347,889 shows a positioning device to facilitate the maintenance of a patent airway by correctly positioning a patient's head under anesthesia during an operation or procedure comprising at least one adjustable support including a plurality of segments or sections selectively positionable relative to adjacent segments or sections and selectively lockable relative to each other having a patient engaging member coupled to the proximal end portion thereof to engage a portion of the patient's head to maintain the position of the patient's head during an operation or procedure. Although this patented device purportedly has the ability to restrain the patient's head, along with provide chin lift and jaw thrust, it has no adjustability in terms of head and/or neck positioning. This device also cannot displace weight off of an obese patient's chest, which can be crucial for air exchange. Furthermore, this device cannot adjust head and neck positioning in real time and it cannot allow for nasal ventilation during intubation.

U.S. Pat. No. 8,001,970 provide devices for use with a patient under anesthesia and associated methods. Various embodiments include a device for establishing and maintaining a patient's head and/or jaw in a particular position, including the sniffing position. Embodiments also include a method for positioning a patient comprising the use of a device of the present invention, wherein the device may substantially maintain a patient in a desired position. Although this patented device has the ability to provide chin lift and jaw thrust, it cannot adjust the head and/or neck to provide custom fit positioning. This device also cannot displace weight off of an obese patient's chest which can be crucial for air exchange. Also this device does not allow for nasal mask ventilation during intubation. It also cannot adjust head and neck positioning while the provider is attempting intubation. And, this patented device also is not able to align all 3 axes in the lateral decubitous position for intubation, nor does it provide a measuring device to confirm the desired position.

U.S. Pat. No. 8,191,553 shows a pair of pads that is held against the ramus of a patient's jaw, to prevent the jaw from slipping back and causing an airway obstruction, while the patient's neck is hyperextended to also cause the patient's airway to stay open. A device including the adjustable jaw pads as well as a triangularly shaped portion over which the patient's neck rests is not required to be attached to the surface on which the patient is lying, and permits the patient to be rolled on either side while still maintaining the patency of the patient's airway. Although this patented device has the ability to provide chin lift and jaw thrust, it cannot adjust the head and/or neck to provide custom fit positioning. This device also cannot displace weight off of an obese patient's chest which can be crucial for air exchange. It also cannot adjust head and neck positioning while the provider is attempting intubation. This patented device also is not able to align all 3 axes in the lateral decubitous position for intubation, nor does it provide a measuring device to confirm the desired position.

U.S. Pat. No. 1,131,802 shows a device comprising a frame having a pair of angular adjustable side extensions, a vertically adjustable head rest detachably mounted on the frame with freedom for horizontal adjustment longitudinally of the side extensions a pair of vertically adjustable jaw rests mounted on the side extensions for movement toward and away from the head rest. The objective of this device was to be used to secure a corpses head to a table for embalming, and thus is not designed to maintain a patent airway, nor to displace weight off of a patient's chest to optimize ventilation.

U.S. Pat. No. 1,441,817, which relates to an apparatus comprising a base plate and a pair of spaced jaw props adjustable on the base plate at an angle thereto. The jaw props includes the sole projections on the base plate, and the base plate being sufficiently narrow so that it may be placed beneath the neck of a corpse and be adjusted longitudinally of the neck of a corpse while the shoulders of the corpse and the head of the corpse rest upon a head board independently of the base plate. The objective of this patent device is to secure a corpse's head to a table for embalming, and is not designed to maintain a patent airway or to displace weight off of a patient's chest to optimize ventilation.

U.S. Pat. No. 1,729,525 teaches a device comprising a vertically adjustable head rest, jaw rests, angularly and lengthwise adjustable supporting means for and carrying the jaw rests carried by the head rest, a supporting structure and head rest having contacting means for latching the head rest in adjusted position, the means including a pair of supports hinged to the head rest and a combined coupling and adjusting device for the supports, the jaw rests being vertically and angularly adjustable with respect to the means, the means further having the forward ends thereof apertured for receiving the jaw rests and carrying at their forward ends clamping devices for maintaining the jaw rests in adjusted position. The objective of this device is to secure a corpse's head to a table for embalming, and is not designed to maintain a patent airway nor to displace weight off of a patient's chest to optimize ventilation.

U.S. Pat. No. 1,776,167 shows a device comprising an adjustable head rest element including a pair of oppositely disposed downwardly inclined extensions and a vertical post, an adjustable supporting element including an angle shaped pivoted arm arranged below the head rest element, the supporting element including means for latching the arm, an adjustable coupling device between the post and the arm, a pair of angle shaped oppositely extending jaw rest elements, adjustable coupling devices between the jaw rest elements and the extensions and adjustable shoulder drawing down means pivotally and adjustably connected to the extensions. This patent does not claim to maintain a patent airway, nor does it claim to achieve the desired position, nor displace weight off of a patient's chest to optimize ventilation. Also this device does not allow for nasal mask ventilation during intubation. It also cannot adjust head and neck positioning while the provider is attempting intubation. The device also does not claim to align all 3 axes (oropharyngeal, laryngeal, tracheal) in the lateral decubitus position for the optimal view for intubation.

U.S. Pat. No. 2,452,816 discloses a jaw supporting device comprising a base member means for securing the base member to and transversely of a table top, abutments upstanding in adjustable spaced opposition from the base member, means for selectively adjusting the abutments longitudinally of the base member. A straight cylindrical stem clampably swiveled to extend upwardly from each abutments upper end, a tubular element telescoped over each stem and slidable axially thereon, means for clamping the tubular element to and in selectively adjusted positions along the stem, a mounting block clampably swiveled to the free end of each tubular element and a jaw engaging cushion removably and replaceably clipped to and in supported relation against each block. The disadvantage to this device is that it requires mandible arms to be engaged in order to achieve the desired position but cannot be used in the lateral decubitous position. This maneuver can be very stimulating and painful to patients that are not deeply anesthetized. Also, it does not provide a mechanism to restrain the head if the patient moves. Obviously if the patient moves the desired position is no longer achieved. This patented device also lacks a way to displace weight off of an obese patient's chest to optimize ventilation, and the patent nowhere teaches or suggests the possibility of aligning all 3 axes in the lateral decubitous position for intubation.

U.S. Pat. No. 4,700,691 relates to a restraining and supporting device for the head of a patient comprising a head immobilizing contraption connected to the operating table, arm and hand supports for the surgeon, wherein the hand supports are fixed to the head immobilizing contraption through flexible arms, also provided with elements releasing or tightening the flexible arms, which elements are fitted to one of the fingers of the surgeon's hand, or interconnected with hand and/or foot switch. The head immobilizing contraption consists of nape support provided with a three-point bearing for the head and can be set at an adjustable height. A front support clamps down the head into the nape support and is connected to the nape support through a hinged mechanism. The flexible arms are attached to the front support of the head immobilizing contraption. The main objective of this device is to restrain the head during surgery, it does not have the capability of maintaining a patent airway, nor does it have the ability to perform a jaw thrust. Also, the device cannot displace weight off of an obese patient's chest to optimize ventilation.

U.S. Pat. No. 5,524,639 discloses an apparatus intended to maintain or improve a supine patient's airway in a hands-free environment. A frame and detachable pillow device are placed under the patient's head. Mechanisms extend laterally from the frame and provide jaw support members that may be brought under the angles of the jaw. The jaw support members may slide towards and away from the frame, but this sliding movement is regulated by a unidirectional clutch, such as a ratchet and pawl system, which restricts the jaw support members to sliding movement away from the frame only. When the jaw support members are slid away from the frame, they engage the angles of the jaw, and then thrust the jaw forward to maintain or improve the patient's airway. Once the desired anteriorly thrust position of the jaw is achieved, the unidirectional clutch holds the jaw in place until the clutch is released. The weight of the jaw then causes the jaw support members to slide back towards the frame, restoring the jaw to its normal position. This device has several disadvantages, the first being that it can only achieve the desired position by using the jaw support members. Not all patients will require jaw support, and since it is very painful and stimulating, trying a lesser invasive approach first would be ideal. Also, this patent does not teach or suggest aligning all 3 axes in the lateral position, and it does not displace weight off of a patient's chest.

### SUMMARY OF THE INVENTION

The present invention in one aspect relates to a positioning device that can either be retro-fit to existing operating room tables or built into future operating room tables to facilitate the maintenance of a patent airway by correctly positioning a patient's neck, head, and torso either while a patient is unconscious, lacks the ability to maintain an open airway, or is under anesthesia during an operation or procedure while the patient is lying on either of their sides. Although several pillows are designed to place the user's head in the sniff position while the user is on his or her back, many procedures require the patient to lay on their side, i.e., the so-called “lateral decubitus position”. Although one pillow currently exists (U.S. patent 2012/0180220) which claims to place a patient in the sniffing position (aligning the three axes, oropharyngeal, tracheal, laryngeal,) when lying on his or her side in the lateral decubitus position, a need exists for a provider to have the option to perform a jaw thrust in a hands free fashion in case emergency situations arise where medications have to be pushed or further management is required. See also U.S. Pat. No. 7,467,431 in which there is described a patient incline device which includes an incline ramp and a centerline spinal support located on a base member. The incline ramp supports the upper torso and head of a patient such that the upper torso and head are elevated with respect to the base member. The centerline support is located adjacent the inline ramp for contact with a central portion of the patient's back located adjacent the spine to elevate the central back portion. According to one embodiment, the incline ramp and the spinal support are adjustable to custom fit to the patient's torso. The width of the spinal support is less than that of the incline ramp to define lateral spaces along opposite sides of the centerline support to receive the arms and the side portions of the patient for lateral extension of the chest wall. See also U.S. Pat. No. 8,336,142.

For a patient in the lateral decubitus position, the present invention also provides an improvement over prior art positioning devices enabling the sniff position, nasal and full-mask non-invasive positive pressure ventilation CPAP, BiPAP, ventilation during intubation, oxygenation during patient transport, jaw thrust, and comprising a base having a first surface for supporting an adjustable ramp and carriage for supporting a patient's back, and a second surface for supporting a patient's head on a second surface which is adjustable on two axis X & Y, to place the patient in a desired sniffing position.

More particularly, the present invention, in one aspect provides a device including a base for supporting a carriage subassembly, the carriage subassembly comprising three surfaces, a first surface, a second surface, and a third surface all adjustable along the Z-axis along the base subassembly. The bases' surfaces each have one side, wherein the base is configured to substantially support the carriage subassembly arm. The carriage subassembly comprises a first surface connected to the second surface and movable along the Z-axis. The first surface of the carriage subassembly comprises two surfaces, a first surface or ramp which assists in maintaining the patient's torso in an inclined position, and a second surface which provides a head-supporting surface for supporting the patient in a desired, i.e., sniffing position.

In another embodiment of the invention, a back restraining device is provided which includes a back board consisting of a rigid surface that comes in contact with the patient's back and is adjustable in the y-axis; a flexible and soft back restraining device having a first proximal end that attaches to one side of the back board, which can then extend horizontally and come in contact with the patient's abdomen and attach to the opposite side of the back board. The device is adjustable and able to secure the patient's back to the back board.

In yet another embodiment of the invention, when the patient is in the lateral decubitus position there is provided a head/neck support that may be independently controlled to support the desired, i.e., sniff position by raising the head and neck independently of the ramp, or if the patient is in either the right or left lateral decubitus position the head and neck supports can be adjusted to ensure proper alignment of the cervical, thoracic, and lumbar vertebra. More particularly, there is provided a pneumatic or mechanical head/neck support that consists of either a mechanical jack or a compliant bellows that is fixed or located on a top surface of head/neck rest.

The present invention also provides methods for positioning a patient. The methods include the steps of: providing a device having a back board and a support arm; placing the patient's head on a first surface of the support arm of the device; adjusting a second surface of the support arm to come in contact with the patient's neck, moving the placing the patient's head and neck in the desired position, along with aligning the cervical, thoracic, and lumbar vertebra to the desired position; restraining the patient's head to prevent the patient from being dislodged from the desired position; moving a first mandible arm to contact the patient's jaw; and moving the second mandible arm to contact the patient's jaw; wherein the contact of the first mandible arm and the second mandible arm provides sufficient force to substantially maintain the patient's head, neck, and/or jaw in a desired position. The back board may then be adjusted to come in contact with the patient's back, and restrain the patient's back to prevent the patient from being dislodged from the desired position.

In yet other embodiment, the present invention includes a mandible arm including: a curved portion that consists of an adjustable and lockable mechanism that attaches to a mandible pad, wherein the mandible pad is flexible, and wherein the mandible pad has a distal side configured to attach to the curved portion and a proximal side configured to contact a patient's jaw at a plurality of points, which can pivot in all angles at the distal end. A connector portion is provided which is configured to attach to a support. Preferably, the connector portion is configured to attach to a support that is attached to a base comprising a left side and a right side, wherein the base is configured to substantially accommodate a patients neck and head, and wherein the support is moveable in three axis such that the mandible pad is positionable to be in contact with the patients jaw at one or more points and to maintain a desired position.

The present invention also provides a method for positioning a patient including the steps of: providing a device having an adjustment mechanism in the z-axis (ie: a which may be a pneumatic jack such as a bellows or a mechanical jack, etc), placing the patient's head substantially on top of the adjustment mechanism, elevating the ramp and then adjusting the adjustment mechanism in the z-axis, as well as a device having an adjustment mechanism in the vertical direction, (ie: a pneumatic jack such as a bellows or a mechanical jack, etc), placing the patient's neck substantially on top of the adjustment mechanism, and then adjusting the adjustment mechanism in the z-axis so that the patient is initially in either a sniff position or an “ear-to-sternal notch position”.

In another aspect the invention provides a method for positioning a patient comprising providing a device as above described placing the patient's head and neck substantially on the first surface of the carriage, and using either one or more adjustable devices to place the patient's head and neck in a desired position. The first mandible arm and second mandible arm are then moved to contact the patient's jaw, wherein the contact of the first mandible arm and the second mandible arm provides sufficient force to substantially maintain the patient's head and/or jaw in a desired sniffing position.

For a patient lying on their back (supine position), in yet another aspect of the invention, there is provided a positioning device to facilitate the maintenance of a patent airway by correctly positioning a patient's back, shoulders, neck and head, while the patient is unconscious, unable to maintain a patent airway, sedated, or under anesthesia during an operation or procedure. The present device includes a device including: a base support comprising a first side, a second side, and an inner adjustable support structure. The first side base is configured to substantially accommodate a patient's neck and head. The distal end of the first base supports the patient's neck and consists of a support adjustable in the z-axis, which assists in maintaining the desired sniffing position. The proximal end of the base consists of a slightly inclined or flat surface adjustable in the z-direction, with or without a cut-out center, which provides head support to further optimize the sniffing position. An adjustable ramp can be placed under the patient's back and shoulders to achieve the desired angles. A first support is positioned on the first side of the base, and a second support positioned on the second side of the base. An adjustable support structure within the interior of the base is provided to mechanically adjust the height and length of the ramp to custom fit to the patient. A first mandible arm is configured to contact the one side of the patient's jaw, and a second mandible arm positioned on the second support, and is configured to contact the other side of the patient's jaw. The first and second support of the mandible arms are moveable on the X, y and z axis. The first and second proximal parts of the moveable arms are moveable along the x, y and z axis, while the distal parts are rotatable, such that each is positionable to be in contact with the patient's jaw, and maintain a desired position.

The present invention also provides an improvement over prior art positioning devices enabling both the sniff position and jaw thrust, and comprising a base having a surface for supporting a carriage for supporting a patient's head adjustable in a Z-axis, supporting a patient's neck on either the first surface or the second surface, which is adjustable in the z-axis, to place the patient in a desired sniffing position. An upper arm constrains the patient's head in translation along all three axes. First and second mandible arms are provided extending from the first surface for contact with the patient's jaw, so as to maintain the patient in a desired position the patient lies on his or her back.

In another aspect the invention provides a method for positioning a patient providing a device as above described placing the patient's head substantially on the first surface of the carriage, and using an adjustable device to place the patient's head and/or neck in a desired position. The first mandible arm and second mandible arm are then moved to contact the patient's jaw, wherein the contact of the first mandible arm and the second mandible arm provides sufficient force either by the provider or mechanically to substantially maintain the patient's head, neck, and/or jaw in a desired sniffing position.

In still yet other embodiments, there is provided a flexible and soft head restraining device either attachable or built in to an anesthesia full face mask, nasal mask, and nasal-oral mask, comprising two ends; a first proximal end that attaches to one side of the first surface of a base; which can then extend horizontally and come in contact with the patient's anesthesia mask, which creates a seal to the patient's nose and/or face and attaches to the opposite side of the first surface of the base; the device is adjustable and able to secure the patient's head and neck to the first surface of the base.

In yet another embodiment, the present invention includes a mandible arm including: a curved portion that consists of an adjustable and lockable mechanism that attach to a mandible pad, wherein the mandible pad is flexible, and wherein the mandible pad has a distal side configured to attach to the curved portion and a proximal side configured to contact a patient's jaw at a plurality of points, which can pivot in all angles at the distal end; and a connector portion which is configured to attach to a support.

A further embodiment to the present invention includes an inclined surface that consists of two sides where the proximal side may attach to the first base, and the angle at which the proximal side is positioned can be changed by adjusting the angle of the distal side (either mechanically, actuation, etc). The distal side also has the ability of extending in order to ensure custom fit head, neck, and torso positioning. The inclined surface will have a back and shoulder pad that rests on it to support a patient's upper back, middle back, and shoulders. This support will enable gravity to displace weight off of patient's chest, allowing for a more patent airway.

And yet another embodiment to the present invention includes a measuring device to confirm the optimal neck flexion angle of 35°. One embodiment consists of two sides, the first side of which is semi cylindrical and consists of four arms, each of which is located within each of the four corners, each of which makes contact with the patient's neck; the second side consists of a 35° leveled angle.

In yet another embodiment the present invention includes a leveling device used to confirm the optimal head extension angle of 15°. This latter embodiment consists of two sides, the first side or which triangular and consists of three arms, each of which is located within each of the three corners, each of which is adjustable in the z-axis, each of which makes contact with the patient's face; the second side consists of a 15° leveled angle.

In another embodiment, the present invention includes a method for positioning a patient including the steps of: providing any embodiment of the devices as described herein, placing the patient's upper back, middle back, and shoulders on an inclined surface along with the patient's neck and head substantially on the base of the device; placing the patient's head and neck in the desired position, optionally confirming the position with a measuring device; securing the anesthesia nasal mask, full face mask, or nasal-oral mask to the patient's nose or face, restraining the patient's head to prevent the patient from being dislodged from the desired position; moving a first and second mandible arm to contact the patient's jaw; wherein the contact of the first mandible arm and the second mandible arm provides sufficient force to substantially maintain the patient's head, neck, and/or jaw in a desired position.

In still yet another aspect of the invention there is provided a simple and elegant adjustable head rest neck rest, or combined head-neck rest, which may be a pneumatic jack such as a bellows, or a mechanical jack, that is independently controllable to support a patient's head and/or neck to obtain an optimal sniff position by raising a patient's neck and head independently of the carriage. The head rest and/or head-neck rest can either be fixed to the device or detachable and moved anywhere on either the device, an operating room table, or any other surface used for patient's requiring airway management. The head rest and/or head-neck rest will also have a cover to protect it from blood, saline, and infectious agents that can also either be fixed to the device and reusable or it can be disposable and detachable. The head rest and/or head-neck rest cover will also have attachments for the mask anchor to attach to and be able to secure the patient's head and/or neck in position, anywhere on the device, operating room table, or on other surface used for patient's requiring airway management. The head rest and/or neck rest cover will also have a second nasal mask, full face mask, or nasal-oral face mask strap that is either reusable and attached to the cover or detachable and disposable and comes from behind the patient's head and attaches to the anesthesia mask in front. This head rest and/or neck rest cover with a mask strap either attached or detachable is novel allows a patient's head and/or neck to remain in the desired position, while the anesthesia mask is sealed to the patient's face anywhere on either the device, operating room table, or on other surface used for patients requiring airway management.

In yet another embodiment the mask strap that is either attached or detached from the head rest and/or neck cover will comprise of a base with one or more sides. The first side can be used to come across the anesthesia mask from the front and attach to the base on the opposite side in order to create a tight seal between either the nasal mask, full face mask, or nasal-oral mask and the patient's face. In yet another embodiment the mask strap will have one side that attaches to the left side of the anesthesia mask, while the second side attaches to the right side or vice versa in order to create a tight seal between either the nasal mask, full face mask, or nasal-oral mask and the patient's face. In yet another embodiment the mask strap will have three sides where one side that attaches to the left side of the anesthesia mask, while the second side attaches to the right side or vice versa and the third side comes over the patient's head attaches to the top part of the anesthesia mask in order to create a tight seal between either the nasal mask, full face mask, or nasal-oral mask and the patient's face.

In yet another aspect of the invention an apparatus for providing anesthesia to a patient is provided including a base comprising a first side, a second side, a third side, and an inner adjustable support structure. The first side of the base is configured to substantially accommodate a patient's neck and head. The distal end of the first base, which supports the patient's neck consists of either a generally semi-cylindrical support, which assists in maintaining the desired sniffing position or a generally flat surface both of which is adjustable in the z-axis. The proximal end of the first base consists of either a slightly inclined or a generally flat surface with or without a cut-out center also adjustable in the z-axis in order to provide head support to further optimize the sniffing position. An adjustable support structure is provided within the interior of the base to mechanically adjust the height of the patient. and custom fit the patient's head, neck and torso to the surface to optimize positioning. The second and third sides of the base each contain a plurality of protrusions, preferably four protrusions. The anesthesia mask attaches to the patient from an anterior perspective, with straps that attach to the mask connecting to the support behind the patients head. Current straps utilize a posteriorly, with the strap-s starting from behind the head and attaching to the mask in front. The mask strap has four sides, sides one and two which contain an aperture that is placed over the aperture of the mask of side one, and sides three and four which contain one narrow extension each of which include a plurality of holes adapted to attach to one of the protrusions on sides two and three of the base. An alternate and preferred design consists of two cords that are attached to the mask, and the cords can then attach to the support behind the head. These cords can be clipped through friction, hook and loop, etc. on each side of the head. The mask strap is adapted to hold the anesthesia mask against either the patient's nose only, nose, mouth, cheeks, and/or or head to maintain the desired sniffing position where the patient's jaw is moved up and forward, thereby preventing a patient's airway from becoming obstructed. The mask strap is adapted to stabilize the patient's head and/or neck to the base preventing movement of the patient's head and/or neck. The tight seal that the mask strap creates also allows for non-invasive positive pressure ventilation (CPAP/BiPAP), which further helps to maintain a patent airway.

In another aspect of the invention, there is provided an apparatus for providing anesthesia to a patient, which comprises, a base having a first side, a second side, a third side, and an adjustable support structure, wherein the base is configured to substantially accommodate a patient's neck and head; wherein a distal end of the base first side comprises either a semi-cylindrical support adjustable in the z-axis, which assists in maintaining the desired sniffing position or a flat surface; a proximal end of the base first side comprises either a slightly inclined or flat surface in order to provide head support to further optimize the sniffing position and is adjustable in the z-axis; the second and third sides of the base can each contain a plurality of protrusions and a mask strap with four sides, wherein the first and second sides include an aperture that is placed over the aperture of the mask, and the third and fourth sides include a narrow extension which include a plurality of holes adapted to attach to one of the four protrusions on sides two and three of the base, or an alternate and preferred design consists clips on each of the second and third side for which the mask cords to attach to; wherein the mask strap is adapted to hold a anesthesia mask strap against the patient's head, mouth and/or nose to maintain the desired sniffing position where the patient's jaw is moved up and forward, thereby preventing the obstruction of the patient's airway; and wherein the mask strap is adapted stabilize the patient's head and neck to the base preventing movement of the patient's head and neck.

Other embodiments of the invention include:

I. A device for positioning a patient, comprising: a base subassembly comprising a surface for supporting a carriage subassembly, where the first surface of the support arm supports the patients head and is adjustable in the Z-axis, the second surface supports the patient's neck and is adjustable in the z-axis, to place the patient in the desired sniffing position; the surface of the upper arm is lowered until comfortably tight and locked, for constraining the patients head in translation along all three axes; a flexible band for placement over the patient's forehead, for securing it to the sniff subassembly by applying a constant constraining force in the −X direction; a first mandible arm extending vertically from the first surface of the carriage subassembly, wherein the first vertically adjusted portion is lockable in rotation about the Z axis, wherein the first mandible arm is positionable to be in contact with the patient's jaw; and a second mandible arm extending vertically from the third surface of the carriage subassembly, wherein the second vertically adjusted portion is lockable in rotation about the Z axis, wherein the second mandible arm is positionable to be in contact with the patient's jaw; wherein the first mandible arm and the second mandible arm are movable such that each is positionable to be in contact with the patient's jaw and to maintain the patient in a desired position while lying on his or her side and leaving the provider hands free.

The above described device preferably is characterized by one or more of the following features

(a) wherein the mandible arm is positionable to be in contact with the patient's jaw at a ramus, a body, or an angle of the patient's jaw while the patient is lying on his or her side, wherein each of the first mandible arm and the second mandible arm preferably is positionable in contact with the patient's jaw at two or more of a ramus, a body, or an angle of the patient's jaw while the patient is lying on his or her side, and/or wherein each of the first mandible arm and the second mandible arm preferably is positionable in contact with a patient's jaw at a ramus, a body, and an angle of the patient's jaw while the patient is lying on his or her side;

(b) further including a mandible pad on the first and second mandible arms, for contact with the patient's jaw;

(c) wherein all surfaces of the carriage subassembly are movable relative to the base, wherein the first and third surfaces of the support arm preferably are movable relative to the base in one axes, and the second surface of the carriage subassembly is movable relative to the base in three axes;

(d) wherein the base is rectangular and the carriage subassembly surfaces are c-shaped;

(e) wherein the mandible pads preferably are formed of a resiliently deformable material, wherein the mandible pads are formed of foam;

(f) wherein the first mandible arm and the second mandible arm are removably connected to the first surface of the carriage subassembly and the third surface of the carriage subassembly, respectively;

(g) wherein the first mandible arm is movable relative to the first surface of the carriage subassembly and the second mandible arm is movable relative to the third surface of the carriage subassembly;

(h) wherein the desired position is the sniffing position while lying on a side, aligning all 3 axes (oropharyngeal, laryngeal, and tracheal), and/or the jaw thrust maneuver;

(i) wherein the second surface of the carriage subassembly further comprises a neck rest disposed to provide optimal flexion of the patient's neck and optimal head extension to a acquire the desired position;

(j) wherein the first and second mandible arms are configured to extend the patient's jaw when rotated in the x-axis;

(k) wherein the plurality of mandible arms are configured to extend the patient's jaw when rotated in the x-axis;

(l) wherein the device is formed of MRI or Xray compatible materials;

(m) wherein the carriage subassembly is reversible, allowing the patient to be placed on an opposite side;

(n) further including a level for determining a patient's neck flexion angle;

(o) wherein the base further comprises a distal neck rest disposed on the first surface configured to provide optimal flexion of the patient's neck and a proximal inclined head rest to provide optimal head extension to provide a desired patient position;

(p) wherein the first and second support surfaces and first and second rotatable portions are adjustable while the patient is in contact with the first and second mandible arms;

(q) wherein the base subassembly comprises:
a rigid inclined side with two ends;
a proximal end which is detachable from the distal side of the first side of the best; the desired position can be obtained by different body habitus' by adjusting the height of the first base;
a distal end comprises an extension mechanism to maintain the desired angle to maintain the patient in the desired position;
a foam pad that lies on top of the rigid inclined side and comes in contact with the patient's upper back, middle back, and shoulders;

(r) wherein the inclined side supports a patient's upper back, middle back, and shoulders, and will enable gravity to displace weight off of the patient's chest, including in obese patients;

(s) wherein the device is formed of MRI or Xray compatible materials.

(t) wherein placing the head and neck substantially on the neck rest on a distal end of the inclined side and a head rest on a proximal end of the inclined side places the patient in the desired position within eliciting pain; and

(u) wherein the device is adjustable along a y-axis to displace weight off of a patient's chest; is adjustable along a y-axis to align the ear and the sternum horizontally to achieve maximal air exchange in obese patients; and is adjustable along a z-axis for elevating and lowering obese patients without the help of health care workers.

II. Also provided is a method for positioning a patient comprising the steps of:

providing the above described device, placing the patient's head substantially on the first surface of the carriage subassembly using an adjustable device to place the patient's head and neck in a desired position; moving the first mandible arm to contact the patient's jaw; moving the second mandible arm to contact the patient's jaw; wherein contact of the first mandible arm and the second mandible arm provides sufficient force to substantially maintain the patient's head and/or jaw in a desired position, wherein all three axes (oropharyngeal, laryngeal, tracheal) preferably are aligned for view for intubation.

III. Also provided is a mandible arm for positioning a patient, comprising: a rigid lockable arm, wherein the arm has a curved extension which is rotatable in the x-axis; a curved portion, wherein the curved portion is substantially rigid; a mandible pad, wherein the mandible pad is flexible and pivotable, and wherein the mandible pad has a proximal side configured to attach to the curved portion and a distal side configured to contact a patient's jaw at a at least two of a ramus, a body, and an angle of the patient's jaw; and a connector portion, wherein the connector portion is configured to extend from and attach to a rotatable portion of a support, and wherein the connector portion is further configured to attach to a support that is attached to the carriage subassembly comprising a left side and a right side, wherein the carriage subassembly is configured to substantially accommodate a patient's head and wherein the carriage subassembly is movable in one axes such that the mandible pad is positionable to be in contact with a patient's jaw at one or more points and to maintain a patient in a desired position, wherein the carriage subassembly is reversible, allowing the patient to be place on an opposite side.

IV. Also provided is a flexible and soft head restraining and anesthesia mask sealing device for positioning a patient, comprising: a first proximal end that attaches to one side of the first side of a base of a carriage subassembly, which can then extend horizontally and come in contact with either the patient's head and attach to the opposite side of the first surface of the base or comes in contact with the anesthesia mask, which then contacts and seals to the patient's face; wherein the device is adjustable and able to secure the patient's head to the first surface of the base to prevent the patient from disengaging from the desired position, wherein the carriage subassembly optionally is reversible, allowing the patient to be placed on an opposite side, and/or wherein the device optionally is formed of MRI or Xray compatible materials.

V. Also provided is a flexible and soft back restraining device for positioning a patient, comprising:

a first proximal end that attaches to one side of the back board, which can then extend horizontally and come in contact with the patient's abdomen and attach to the opposite side of the back board, wherein the device is adjustable and able to secure the patient's back to the surface of the back board to prevent the patient from disengaging from the desired position.

VI. Also provided is a surface for supporting a patient, comprising: a first side that is adjustable to be in contact the patient's back, and a second adjustable side that is adjustable to be in contact with the patient's ribs to prevent patient movement.

VII. Also provided is an apparatus for use when providing anesthesia to a patient, comprising:

a base having a first side, a second side, a third side, and an adjustable support structure, wherein the base is configured to substantially accommodate a patient's neck and head; wherein a distal end of the base first side comprises either a semi-cylindrical support, adjustable in the z-axis, which assists in maintaining the desired sniffing position or a flat surface also adjustable in the z axis; a proximal end of the base first side comprises either a slightly inclined or flat surface, with or without a cut-out center and is adjustable in the z-axis in order to provide head support to further optimize the sniffing position; the second and third sides of the base each contain a plurality of protrusions; and an anesthesia mask strap with four sides, wherein the first and second sides include an aperture that is placed over an aperture of the mask, and the third and fourth sides include a narrow extension which include a plurality of holes adapted to attach to one of the four protrusions on sides two and thereof the base; wherein the mask strap is adapted to hold an anesthesia mask strap against the patient's nose, cheeks, mouth and/or head to maintain the desired sniffing position where the patient's jaw is moved up and forward, thereby unobstructing the patient's airway; and wherein the mask strap is adapted stabilize the patient's head and neck to the base preventing movement of the patient's head and neck.

The above described apparatus preferably is characterized by one or more of the following features:

(a) wherein the anesthesia mask strap is formed of a non-static latex free material;

(b) wherein at least some of said narrow extensions have respective portions of snap fasteners for attaching the narrow extension to one of the protrusions of the base;

(c) wherein the adjustable support is located in the interior of the base;

(d) wherein the adjustable support includes a mechanical or a pneumatic adjustment mechanism;

(e) wherein the base is substantially rectangular in plan;

(f) wherein the desired position is the sniffing position, aligning all three axes, oropharyngeal, laryngeal, and tracheal;

(g) wherein the base further comprises a distal adjustable neck rest disposed on the first surface configured to provide optimal flexion of the patient's neck and a proximal inclined head rest to provide optimal head extension to acquire the desired position; or the base comprises only a flat surface if the desired positioned is not necessary;

(h) wherein the apparatus is formed of MRI or Xray compatible materials; and

(i) wherein the mask strap is formed of a material that is easily disinfected with anti-microbial solutions or is disposable.

VIII. Also provided is a method for positioning a patient for administering anesthesia, comprising the steps of: providing the apparatus above described, placing the patient's head and neck substantially on the carriage subassembly; using an adjustable device to place the patient's head and neck in a desired position; placing the mask strap either over the aperture of the anesthesia mask or the patient's head to substantially maintain the patient's head and/or jaw in a desired position.

The above method preferably is characterized by one or more of the following features:

(a) wherein placing the head and neck substantially on the neck rest on the distal end of the first surface of the base and the head rest on the proximal end of the first side of the base places the patient in the desired position without eliciting pain;

(b) wherein placing the head and neck substantially on the neck rest on the distal end of the first surface of the base and the head rest on the proximal end of the first side of the base places the patient in the desired position and restricts the movement of the patient's head and neck; and

(c) wherein placing the mask on the patient's face, then placing the mask strap over the mask and attaching it to the base will prevent leakage of anesthetic gases and oxygen into the air.

IX. Also provided is an apparatus for use when providing anesthesia to a patient, comprising: means for providing said anesthesia to said patient; mask strap means for holding said mask means against a patient's nose, mouth, cheeks, and or head; a base of which the mask strap attaches to achieve the desired position which raises the patient's jaw up and forward so as to unobstruct the patient's airway.

X. Also provided is an apparatus for providing ventilation to a patient lying supine on a support, comprising, a ventilation mask, a mask anchor ring over the ventilation mask, and

a plurality of elastomeric straps connecting the mask anchor to the support.

Preferably elastomeric straps are fixed to the mask anchor ring spaced 180° around an imaginary circle.

XI. Also provided is a device for positioning a patient, comprising: a carriage having a first surface that supports the patients in an inclined position, and is adjustable in the Z-axis, a second surface that supports the patient's head and neck and is adjustable to place the patient in a generally desired sniffing position; and a pneumatic or mechanical jack, or an expandable bellows, supported on the second surface for independently raising the patient's head relative to the second surface.

The above device preferably is characterized by one or more of the following features:

(a) wherein the expandable bellows comprises a plurality of rigid concentric rings joined by flexible membranes on a rigid base, wherein the third side of the base preferably comprises:

a rigid inclined side with two ends;

a proximal end which is detachable from the distal side of the first side of the best; the desired position can be obtained by different body habitus' by adjusting the height of the first base;

a distal end comprises an extension mechanism to maintain the desired angle to maintain the patient in the desired position;

a resiliently deformable pad that lies on top of the rigid inclined side and comes in contact with the patient's upper back, middle back, and shoulders;

(b) wherein the bellows includes a two-way valve through which air may be added or subtracted; and

(c) wherein the bellows is formed of MRI or Xray compatible material.

XII. Also provided is a method for positioning a patient to facilitate maintenance of a patient airway under anesthesia, comprising providing a device as above described, positioning the patient on the device, adjusting the first surface to support the patient in a desired inclined position; adjusting the second surface to support the patient's head and neck in a generally desired sniffing position; and activating the pneumatic or mechanical jack, or inflating the expandable bellows to raise the patient's head relative to the second surface to a desired sniffing position.

XIII. Also provided is a device for positioning a patient, comprising: a base comprising a first side which supports the patients head and neck, a second side acting as the foundation, an inner vertically adjustable support structure between the first and second sides, and a detachable third inclined side which support the upper back, middle back, and shoulders of patient; a first support positioned on the second side of the base and lockably adjustable with respect to the second side of the base in an x and y axes; a second support positioned on the second side of the base and lockably adjustable with respect to the second side of the base in the x and y axes; a first mandible arm extending from a first vertically adjusted portion of the first support, wherein the first vertically adjusted portion is lockable in a z axis to lockably adjust the first mandible arm with respect to the z axis, and wherein the first mandible arm is positionable to be in contact with the patient's jaw; and a second mandible arm extending from a second vertically adjusted portion of the second support, wherein the second vertically adjusted portion is lockable in the z axis to adjust the second mandible arm with respect to the z axis, and wherein the second mandible arm is positionable to be in contact with the patient's jaw; wherein the first mandible arm and the second mandible arm are movable such that each is positionable to be in contact with the patient's jaw and to maintain the patient in a desired position and leaving the provider hands free.

The above device preferably is characterized by one or more of the following features:

(a) wherein the mandible arm is positionable to be in contact with the patient's jaw at a ramus, a body, or an angle of the patient's jaw, wherein each of the first mandible arm and the second mandible arm preferably is positionable such that the mandible pad, preferably formed of foam, is in contact with the patient's jaw at two or more of a ramus, a body, or an angle of the patient's jaw, or wherein each of the first mandible arm and the second mandible arm preferably is positionable such that the mandible pad is in contact with a patient's jaw at a ramus, a body, and an angle of the patient's jaw;

(b) wherein the first support is movable relative to the base and the second support is movable relative to the base, wherein the first support preferable is movable relative to the base in two axes and the second support is movable relative to the base in two axes;

(c) wherein the base is rectangular;

(d) wherein the first mandible arm and the second mandible arm each comprise a mandible pad;

(e) wherein the first mandible arm and the second mandible arm are removably connected to the first support and the second support, respectively;

(f) wherein the first mandible arm is movable relative to the first support and the second mandible arm is movable relative to the second support;

(g) wherein the desired position is the sniffing position, aligning all 3 axes (oropharyngeal, laryngeal, and tracheal), and/or the jaw thrust maneuver;

(h) further including a level for determining a patient's neck flexion angle;

(i) wherein the base further comprises a distal neck rest disposed on the first surface configured to provide optimal flexion of the patient's neck and a proximal inclined head rest to provide optimal head extension to a acquire the desired position;

(j) wherein the first and second mandible arms are configured to extend the patient's jaw when rotated in the z-axis;

(k) wherein the plurality of mandible arms are configured to extend the patient's jaw when rotated in the z-axis;

(l) wherein the first and second supports and first and second rotatable portions are adjustable while the patient is in contact with the first and second mandible arms; and

(m) wherein the third side of the base will support a patient's upper back, middle back, and shoulders, whereby to enable gravity to displace weight off of the patient's chest, including in obese patients;

(n) wherein the device is formed of MRI or Xray compatible materials;

(o) wherein placing the head and neck substantially on the neck rest on the distal end of the first surface of the base and the head rest on the proximal end of the first side of the base places the patient in the desired position within eliciting pain; and

(p) wherein the inner adjustable surface consists of a rigid structure; wherein the device is adjustable along a y-axis to displace weight off of a patient's chest; is adjustable along a y-axis to align the ear and the sternum horizontally to achieve maximal air exchange in obese patients; and is adjustable along a z-axis for elevating and lowering obese patients without the help of health care workers;

(q) further comprising a claw for providing an anesthesiologist a tactile interface with the patient in terms of extending the jaw, wherein the position of left and right arms of the claw are maintained by frictional force that is transmitted through the jack assembly, which force may be overcome by the anesthesiologist when rotating the arms about the Z axis, further optionally characterized by one or more of the following features:
(i) wherein the position of the left and right arms of the claw are secured by friction about the Y axis;
(ii) wherein a fine adjustment for further extending the jaw is provided by a screw which applies additional force in the nominal Z direction by applying a torque to the arms about the Y axis; and
(iii) further comprising a torque limiter for limiting force applied to the mandible by the left and right arms in the Z direction to prevent injury to the patient.

XIV. Also provided is a mandible arm for use in positioning a patient, comprising: two rigid lockable arms, wherein the upper arm has a curved extension which is rotatable in the z-axis and the lower arm does not provide an extension; a curved portion, wherein the curved portion is substantially rigid; a mandible pad, wherein the mandible pad is flexible and pivotable, and wherein the mandible pad has a proximal side configured to attach to the curved portion and a distal side configured to contact a patient's jaw at a at least two of a ramus, a body, and an angle of the patient's jaw; and a connector portion, wherein the connector portion is configured to extend from and attach to a rotatable portion of a support, and wherein the connector portion is further configured to attach to a support that is attached to a base comprising a left side and a right side, wherein the base is configured to substantially accommodate a patient's head, neck, upper and middle back, and shoulders, and wherein the support is movable in two axes such that the mandible pad is positionable to be in contact with a patient's jaw at one or more points and to maintain a patient in a desired position.

XV. Also provided is a first measuring device for use with the device of as above described, comprising two sides: a first rigid semi-cylindrical side with four arm extensions, each of which is located within each of the four corners, and each of which comes into contact with the patient's neck; a second rigid side consists of a 35° incline, of which rests a measuring device used to confirm the neck flexion angle of 35° to achieve the desired position.

XVI. Also provided is a second measuring device for use with the device as above described, comprising two sides: a first rigid triangular side with three-arm extensions, each of which is located within each of the three corners, and each of which comes in contact with the patient's head; the arm extensions are each adjustable along the z-axis to achieve the desired position; a second rigid side consists of a 15° incline, of which rests a measuring device used to confirm the head extension angle of 15° to achieve the desired position.

XVII. Also provided is a flexible and soft head restraining device for a patient, comprising:

a first proximal end that attaches to one side of the first side of the base; which can then extend horizontally and come in contact with the patient's head and attach to the opposite side of the first surface of the base; the device is adjustable and able to secure the patient's head to the first surface of the base to prevent the patient from disengaging from the desired position.

XVIII. Also provided is a method for positioning a patient comprising the steps of:

providing a device as described above, placing the patient's head, neck, upper and middle back, and shoulders substantially on the base; using an adjustable device to place the patient's head, neck, upper and middle back, and shoulders in a desired position; optionally using a measuring device to confirm the desired position; moving the first mandible arm to contact the patient's jaw; moving the second mandible arm to contact the patient's jaw; wherein the contact of the first mandible arm and the second mandible arm provides sufficient force to substantially maintain the patient's head and/or jaw in a desired position.

The above method preferably is characterized by one or more of the following features:

(a) wherein all three axes (oropharyngeal, laryngeal, tracheal) are aligned for the recommended view for intubation;

(b) wherein the patient's head height is adjusted with respect to the Z axis by using a jack, and

(c) wherein the device includes a squeeze released jaw thrust grip, and including the step of moving the jaw thrust grip in the z direction.

XIX. Also provided is a device for positioning a patient, comprising: a base; a ramp subassembly pivotally mounted at a proximal end to the base for supporting the upper back, middle back, and shoulders of the patient, said ramp subassembly being adjustable in length and angle relative to the base; a head support subassembly pivotally mounted to a distal end of the ramp subassembly, said head support subassembly being adjustable in angle relative to the ramp substantially; and a pneumatic or mechanical jack, or an expandable bellows, supported on the head support subassembly for independently raising the patient's head relative to the head support subassembly.

XX. Also provided is a first measuring device for use with the device as above described, comprising two sides: a first rigid semi-cylindrical side with four arm extensions, each of which is located within each of the four corners, and each of which comes into contact with the patient's neck; a second rigid side consists of a 35° incline, of which rests a measuring device used to confirm the neck flexion angle of 35° to achieve the desired position.

XXI. Also provided is a second measuring device for use with the device as above described, comprising two sides: a first rigid triangular side with three-arm extensions, each of which is located within each of the three corners, and each of which comes in contact with the patient's head; the arm extensions are each adjustable along the z-axis to achieve the desired position; a second rigid side consists of a 15° incline, of which rests a measuring device used to confirm the head extension angle of 15° to achieve the desired position.

XXII. Also provided is a method for positioning a patient comprising the steps of:

providing the above described device; placing the patient's head, neck, upper and middle back, and shoulders substantially on the base; using an adjustable device to place the patient's head, neck, upper and middle back, and shoulders in a desired position; using a measuring device to confirm the desired position; moving the first mandible arm to contact the patient's jaw; moving the second mandible arm to contact the patient's jaw; wherein the contact of the first mandible arm and the second mandible arm provides sufficient force to substantially maintain the patient's head and/or jaw in a desired position.

The above method preferably is characterized by one or more of the following features:

(a) wherein all three axes (oropharyngeal, laryngeal, tracheal) are aligned for the recommended view for intubation;

(b) wherein the patient's head height is adjusted with respect to the Z axis by using a jack;

(c) wherein the device includes a squeeze released jaw thrust grip, and including the step of moving the jaw thrust grip in the z direction;

(d) including a neck interface and a head interface which are independently adjustable in one or more of the x, y and z positions, and

(e) wherein the detachable third incline side is rotatably adjustable about each y axis.

XXIII. Also provided is a method for positioning a patient to facilitate maintenance of a patent airway under anesthesia, comprising: providing a device as above described: positioning the patient on the device; adjusting the ramp subassembly to support the patient in a desired inclined position; adjusting the head subassembly to support the patient's head and neck in a generally desired sniffing position; and activating the pneumatic or mechanical jack, or inflating the expandable bellows to raise the patient's head relative to the second surface to a desired sniffing position.

XXIV. Also provided is a mandible arm for use in positioning a patient, comprising: two rigid lockable arms, wherein the upper arm has a curved extension which is rotatable in the z-axis and the lower arm does not provide an extension; a curved portion, wherein the curved portion is substantially rigid; a mandible pad, wherein the mandible pad is flexible and pivotable, and wherein the mandible pad has a proximal side configured to attach to the curved portion and a distal side configured to contact a patient's jaw at a at least two of a ramus, a body, and an angle of the patient's jaw; and a connector portion, wherein the connector portion is configured to extend from and attach to a rotatable portion of a support, and wherein the connector portion is further configured to attach to a support that is attached to a base comprising a left side and a right side, wherein the base is configured to substantially accommodate a patient's head, neck, upper and middle back, and shoulders, and wherein the support is movable in two axes such that the mandible pad is positionable to be in contact with a patient's jaw at one or more points and to maintain a patient in a desired position.

XXV. Also provided is a flexible and soft head restraining device for a patient, comprising:

a first proximal end that attaches to one side of the first side of the base; which can then extend horizontally and come in contact with the patient's head and attach to the opposite side of the first surface of the base; the device is adjustable and able to secure the patient's head to the first surface of the base to prevent the patient from disengaging from the desired position.

XXVI. Also provided is a device for positioning a patient in a sniff position, comprising an adjustable ramp and headrest, wherein as ramp incline is varied, head rest orientation remains parallel, substantially horizontal to the operating table, by changing the head rest angle, θ_{X} by an opposite amount.

The above device preferably is characterized by one or more of the following features:

(a) wherein adjustment of angles is accomplished by open loop processing based on known or estimated geometries of all known parameters;

(b) wherein adjustment of angles is accomplished by closed loop processing where a current angle is measured relative to an initial angle, and driven back to said initial angle;

(c) wherein adjustment is accomplished upon multiple feedback sensors including but not limited to:
(i) Measurement of angle relative to gravity with an inclinometer; and
(ii) Encoders.

(d) wherein as ramp incline is varied, and/or head rest angle, □ are changed to position the patient, ramp linkage length is varied in order to satisfy the conditions that positions of linkages fixed relative to their respective support surfaces;

(e) wherein adjusting of angles is accomplished by:
(i) Open loop processing based on known or estimated geometries of all parameters, or by
(ii) Closed loop processing where the relative positions of one or more linkage termination points are measured and the length d is adjusted under closed loop control driven by sensor feedback to return the measured parameter to their original position with regard to the following geometry, or by with reference to FIG. 34 appended herewith, and whereas,
(a) Point 2 of linkage a's position relative to point 1;
(b) Point 3 of linkage c's position relative to point 4;
(c) Alternate Linkage Axis Point 3 where the patient head meets the head rest; and
(d) feedback sensors monitoring relative position of the points that define the linkage length including position measurement sensors selected from the group consisting of: hall effect sensors; magneto-resistive sensors; optical sensors, including encoders, interferometers and/or positional sensing detectors; and stress/strain/force/torque monitoring sensors located at the point interfaces that minimize those parameters by adjusting linkage length d under closed loop control.

XXVII. Also provided is a disposable anesthesia nasal and oral mask which can be used either separately as a nasal mask or a oral mask or can be attached together and can be used as a combination nasal-oral mask, which can also be used to sealingly connect a mask to a wearer's face; two cushions comprising: a first nasal inflatable or non-inflatable cushion that consists of a nasal bridge region, a cheek region, and an upper lip region and a second mouth inflatable or non-inflatable cushion which consists of a lower lip region, a cheek region, and an upper lip region; a first nasal membrane comprising a substantially triangularly shaped frame of resilient material having a first molded inwardly curved rim of said first nasal membrane; a second nasal membrane of resilient material, said second nasal membrane being thinner, as thin, or thicker than said first nasal membrane, said second nasal membrane having a second molded inwardly curved rim, said second nasal membrane curved rim spaced a first distance from said first nasal membrane curved rim in said cheek region and said second nasal membrane curved rim spaced a second distance from said first nasal membrane curved rim in said nasal bridge region, said second distance greater than said first distance, said distances measured when the mask is not in use, a portion of said second membrane curved rim forming a face contacting seal; a first mouth membrane comprising a substantially oval shaped frame of resilient material having a first molded inwardly curved rim of said first mouth membrane; a second mouth membrane of resilient material, said second mouth membrane being thinner, as thin, or thicker than said first mouth membrane, said second mouth membrane having a second molded inwardly curved rim, said second mouth membrane curved rim spaced a first distance from said first mouth membrane curved rim in said cheek region and said second mouth membrane curved rim spaced a second distance from said first mouth membrane curved rim in said mouth region, said second distance greater than said first distance, said distances measured when the mask is not in use, a portion of said second membrane curved rim forming a face contacting seal; an attachment with two apertures; where the first aperture is fixed to the oral mask and can connect to the second aperture, which is fixed to the nasal mask; where when they are connected together it comprises an anesthesia full face mask, covering and sealing around the mouth and nose; yet either the mouth mask or the nasal mask can detach so that the mask can be used for nasal non-invasive positive pressure ventilation (CPAP/BiPAP) alone or oral non-invasive positive pressure ventilation (CPAP/BiPAP) alone.

An above described nasal and oral mask preferably is characterized by one or more of the following features:

(a) wherein said second molded rim and said first molded rim have a co-located notch to accommodate the bridge of a wearer's nose;

(b) wherein said first nasal membrane molded rim and said second nasal membrane molded rim are substantially saddle-shaped;

(c) wherein said second nasal membrane is shaped so that said seal portion, in use, contacts at least a wearer's nose;

(d) wherein said seal portion, in use, contacts the facial tissue around the sides and over the bridge of the nose, and between the base of the nose and the top lip;

(e) wherein said second rim and seal portion are shaped to generally match facial contours in the region of facial tissue around the sides and over the bridge of the nose, and between the base of the nose and the upper lip;

(f) wherein the first and second nasal membranes comprise one molded piece, without being adhered together by an adhesive.

(g) wherein the first molded inwardly curved rim of said first nasal membrane is as thick, less thick, or thicker than the second nasal membrane;

(h) wherein the second molded inwardly curved rim of the second nasal membrane is as thick, less thick, or thicker than the first nasal membrane;

(i) wherein said second molded rim and said first molded rim have a co-located notch to accommodate the lips a wearer's mouth;

(j) wherein said first mouth membrane molded rim and said second mouth membrane molded rim are substantially oval shaped;

(k) wherein said second mouth membrane is shaped so that said seal portion, in use, contacts at least a wearer's upper and lower lip;

(l) wherein said seal portion, in use, contacts the facial tissue around the sides and over the upper and lower lips of the mouth;

(m) wherein said second rim and seal portion are shaped to generally match facial contours in the region of facial tissue around the sides and over the upper and lower lip of the mouth;

(n) wherein the first and second mouth membranes comprise one molded piece, without being adhered together by an adhesive;

(o) wherein the first molded inwardly curved rim of said first mouth membrane is as thick, less thick, or thicker than the second mouth membrane, and

(p) wherein the second molded inwardly curved rim of the second mouth membrane is as thick, less thick, or thicker than the first mouth membrane.

XXIII. Also provided is a nasal mask, oral mask or full face mask, for connection to a wearer's face comprising: a mask body for connection with a supply of breathable gas, whether oxygen, air, anesthetic gases or any other gas; and a nasal inflatable or non-inflatable cushion secured to said mask body, the body and cushion forming a nose-receiving cavity, said cushion including: a nasal bridge region, a cheek region and an upper lip region; a substantially triangularly-shaped first nasal membrane of resilient material having a first molded inwardly curved rim to surround wearer's nose; a second nasal membrane also of resilient material, said second membrane being relatively more flexible than said first nasal membrane, said second nasal membrane having a second molded inwardly curved rim, said second molded rim being of the same general shape as said first molded rim and fixed to and extending away from said first nasal membrane so as to have a second nasal membrane inner surface spaced a first distance from an outer surface of said first molded rim in said cheek region and said second membrane inner surface spaced a second distance from said first nasal membrane outer surface of said first molded rim in said nasal bridge region, said second distance greater than said first distance, said distances measured when the mask is not in use, a portion of said second molded rim forming a face contacting seal; and wherein said seal portion is substantially coterminous with respect to said second molded rim and is resiliently deformable towards said first nasal membrane in use of said mask.

The above described nasal mask, oral mask, or full face mask, covering and sealing the mouth and nose, preferably is characterized by one or more of the following features:

(a) wherein said a nasal mask, oral mask, or full face mask body includes either integrated head strap attachment points using either an anterior approach or posterior approach or it can have separated head strap attachment points using either an anterior approach or a posterior approach that placed over the nasal mask, oral mask, or full face mask body, which attach to a surface that can secure the nasal mask, oral mask, or full face mask to the wearer's face to ensure a tight seal and to maintain the wearer's head and neck in the desired position to maintain airway patentcy;

(b) further comprising securing straps fixed to said attachment points which can secure the wearer's head to a surface and maintain the wearer's head and neck in the desired position;

(c) wherein said second membrane molded rim and said first nasal membrane molded rim each have a co-located notch to accommodate the bridge of a nose;

(d) wherein said first and second molded rims are substantially saddle-shaped;

(e) wherein said second nasal membrane is shaped so that said seal portion, in use, contacts at least wearer's nose;

(f) wherein said seal portion, in use, contacts the facial tissue around the sides and over the bridge of the nose, and between the base of the nose and the upper lip, and

(g) wherein said rim and said seal portion are shaped to generally match facial contours in the region of facial tissue around the sides and over the bridge of the nose, and between the base of the nose and the upper lip.

XXIX. Also provided is a nasal noninvasive positive pressure ventilating (CPAP/BiPAP), oral noninvasive positive pressure ventilating (CPAP/BiPAP), or full face mask noninvasive positive pressure ventilating (CPAP/BiPAP) treatment apparatus comprising: a generator for the supply of gas at a pressure below, equal to, or elevated above atmospheric pressure; a gas delivery conduit coupled to said generator; and a nasal mask oral mask, full face mask in turn coupled to said conduit to said nasal mask, oral mask, full face mask including: a mask body for connection with a supply of breathable gas; and a nasal inflatable or non-inflatable cushion secured to said mask body, the body and cushion forming a nose-receiving cavity, the cushion including: a nasal bridge region, a cheek region and a lip region; a substantially triangularly-shaped first nasal membrane of resilient material having a first nasal membrane having a molded inwardly curved rim; a second membrane having a second molded inwardly curved rim also of resilient material, said second nasal membrane being relatively more flexible than said first membrane, and being of the same general shape as said first molded inwardly curved rim and fixed to and extending away from said first nasal membrane so as to have an inner surface spaced a first distance from said first molded rim in said cheek region and said second nasal membrane inner surface spaced a second distance from said first molded rim, said second distance greater than said first distance, said distances measured when the mask is not in use, a portion of said second molded rim forming a face contacting seal; and Wherein said seal portion is generally coterminous with respect to said second molded rim and is resiliently deformable towards said first membrane in use of said mask.

The above described non-invasive positive pressure ventilation (BiPAP/CPAP) treatment apparatus preferably is characterized by one or more of the following features:

(a) wherein said mask body includes attachment points which can secure the wearer's head to a surface and maintain the wearer's head and neck in position;

(b) further comprising securing straps fixed to said attachment points which can secure the wearer's head to a surface and maintain the wearer's head and neck in position;

(c) wherein said first and second molded rims each have a co-located notch to accommodate the bridge of a nose;

(d) wherein said first and second molded rims are substantially saddle-shaped;

(e) wherein said second nasal membrane is shaped so that said seal portion, in use, contacts at least wearer's nose;

(f) wherein said seal portion, in use, contacts the facial tissue around the sides and over the bridge of the nose, and facial tissue around the sides and over the bridge of the nose, between the base of the nose and the upper lip and between the base of the nose and the upper lip, and

(g) wherein said second molded rim and said seal portion are shaped to generally match facial contours in the region of facial tissue around the sides and over the bridge of the nose, between the base of the nose and the upper lip and between the base of the nose and the upper lip.

XXX. Also provided is an oral mask for connection to a wearer's face comprising: a mask body for connection with a supply of breathable gas; and an inflatable or non-inflatable mouth cushion secured to said mask body, the body and cushion forming a mouth-receiving cavity, said cushion including: a mouth region, a cheek region and an upper and lower lip region; a substantially oval-shaped first mouth membrane of resilient material having a first molded inwardly curved rim to surround a wearer's nose; a second mouth membrane also of resilient material, said second mouth membrane being relatively more flexible than said first mouth membrane, said second mouth membrane having a second molded inwardly curved rim, said second molded rim being of the same general shape as said first molded rim and fixed to and extending away from said first mouth membrane so as to have a second mouth membrane inner surface spaced a first distance from an outer surface of said first molded rim in said cheek region and said second mouth membrane inner surface spaced a second distance from said first mouth membrane outer surface of said first molded rim in said mouth region, said second distance greater than said first distance, said distances measured when the mask is not in use, a portion of said second molded rim forming a face contacting seal; and wherein said seal portion is substantially coterminous with respect to said second molded rim and is resiliently deformable towards said first mouth membrane in use of said mask.

The above described mask preferably is characterized by one or more of the following features:

(a) wherein said mask body includes attachment points which can secure the wearer's head to a surface and maintain the wearer's head and neck in position;

(b) further comprising securing straps fixed to said attachment points which can secure the wearer's head to a surface and maintain the wearer's head and neck in position;

(c) wherein said second membrane molded rim and said first mouth membrane molded rim each have a co-located notch to accommodate the mouth;

(d) wherein said first and second molded rims are substantially oval-shaped;

(e) wherein said second mouth membrane is shaped so that said seal portion, in use, contacts at least wearer's mouth;

(f) wherein said seal portion, in use, contacts the facial tissue around the sides and over the mouth, and between the upper and lower lip;

(g) wherein said rim and said seal portion are shaped to generally match facial contours in the region of facial tissue around the sides and the mouth, and between the upper and lower lip.

The above described nasal mask, oral mask, or full facemask, further preferably comprises tubing which has two ends to be used as an gas source to transport patients, where a distal end of the tubing is connected to either a stand alone or a portable generator for the supply of gas at a pressure below, equal to, or elevated above atmospheric pressure; a gas delivery conduit coupled to said generator a portable gas supply and a proximal end is connected to an adaptor, which contains an End-Tidal CO2 port, a nebulizer port, a PEEP valve port, expiratory port and/or valve, pressure relief valve, which has an aperture which attaches to either the nasal mask, the oral mask, or the full face mask.

The above described nasal mask, oral mask or full face mask, also preferably may be connected to a generator for the supply of gas, where the amount and concentration of gas delivered is controlled by the supply source as well as the expiratory port, and/or used as a scavenger system by connecting the nasal mask and the oral mask simultaneously, where the nasal mask can be used to deliver positive pressure and the oral mask can be connected to a suctioning device to properly store and/or dispose gases.

The above described nasal mask, oral mask or full face mask also preferably is contoured around the patient's nasal bridge, nose, and upper lip such that it and the generator gas supply it is connected to does not interfere with the operator's access to the mouth/oral cavity, lips, cheeks, chin, jaw, and neck, and/or connected to a resuscitator bag with or without a gas supply attached to the resuscitator bag.

XXXI. Also provided is an operating table having a positioning device as above described, and one or more pads having a thickness approximating that of the positioning device, on the operating table.

In the above described operating table preferably at least one of the pads is slatted or pleated to facilitate bending. Also, the above described operating table preferably further includes a base spacer having a plurality of rollers located under a main pad.

### BRIEF DESCRIPTION OF THE DRAWINGS

Further features and advantages of the present invention will be seen by the following detailed description, taking in conjunction with the accompanying drawings, wherein:

FIG. 1 is a side elevational view showing a lateral positioning device in accordance with one embodiment of the present invention and illustrates a patient lying on his or her side (lateral decubitus position) in order to displace weight off his or her chest to assist in ventilation; and

FIG. 2 is the lateral device with x, y, and z views in the lateral decubitus position.

FIGS. 3A and 3B diagrammatically illustrate the apparatus and method for positioning a patient in accordance with the present invention for the supine position.

FIGS. 3A-3C are front and rear perspective views and side elevational views of yet another embodiment, the supine positioning device in accordance with the present invention;

FIGS. 4A and 4B are top perspective and side elevational views of the current embodiment shown in a lowered position;

FIGS. 5 and 6 is a side elevational view of the FIG. 4A-4B positioning device retrofitted to existing operating tables in the raised and lowered positions respectively;

FIGS. 7A and 7B are views similar to FIG. 6, of an alternative embodiment of the invention showing the head and neck independent supports mounted on the lift support;

FIG. 8 shows a traditional patient mask strap in accordance with the prior art;

FIGS. 9A and 9B show a mask strap in accordance with an embodiment of the present invention;

FIGS. 10-12 show another embodiment of mask strap in accordance with the present invention;

FIG. 13 shows a head restraint in accordance with the present invention;

FIGS. 14A-14C show a mask anchor ring in accordance with the present invention;

FIG. 14D shows a ring part of the mask and how the posterior straps attach;

FIG. 14D shows a ring part of the mask and how posterior straps will attach;

FIGS. 15A-15C illustrate use of a mask anchor ring in accordance with the present invention, and FIG. 15D illustrates a mask in which the mask anchor ring or mask anchor straps are built into the mask;

FIGS. 15E and 15F are top and bottom plan views of yet another aspect of mask in accordance with the present invention;

FIGS. 16A-16C illustrate a pneumatic head or neck rest in accordance with the present invention;

FIG. 16D illustrates a patient whose head is restrained by an anterior strap.

FIGS. 17A, 17B, 17C, and 17D and 18A and 18B show details of a pneumatic head or neck rest in accordance with the present invention;

FIG. 19 is a flow diagram in accordance with one embodiment of the present invention;

FIGS. 20A, 20B22, and 23 show one embodiment of the jaw claw and ramp subassembly in accordance with the present invention;

FIGS. 21A and 21B and 24A-24D illustrate use of the jaw claw in accordance with the present invention;

FIG. 25 is a flow diagram showing the steps for using the jaw claw in accordance with the present invention, taken in conjunction with FIG. 24 and FIG. 26;

FIGS. 27 and 28A and 28B illustrate a mandible structural model, and FIG. 28C shows a skull and mandible coordinate systems on a device in accordance with the present invention;

FIGS. 29A-29C diagrammatically illustrate a pressure sensing array in accordance with the present invention;

FIGS. 30A-30B and 31A-31C provide additional details of jaw thrust in accordance with the present invention;

FIGS. 32A-32D, 33A and 33B illustrate neck and head positioning adjustment capabilities of the device of the present invention;

FIG. 34 schematically illustrates a four-bar linkage geometry of the lift mechanism of the present invention;

FIGS. 35A-35C and 36A-36F diagrammatically illustrate the lift mechanism in accordance with the present invention;

FIG. 37 plots linkage lengths, ramp angle and head support angle in accordance with the present invention;

FIG. 38 shows a patient in a sniffing position with the jaw thrust device in accordance with the present invention;

FIG. 39 is a flow diagram of the use of the device, in order to maintain coincident neck and neck support locations at linkage axis 4 in accordance with the present invention;

FIGS. 40A-40D and 41 illustrate combined nasal and mouth ventilation masks in accordance with yet another embodiment of the present invention; and

FIGS. 42-45 and 46A-46C illustrate a preferred embodiment of the invention, installed on a conventional operating table.

### DETAILED DESCRIPTION OF THE DRAWINGS

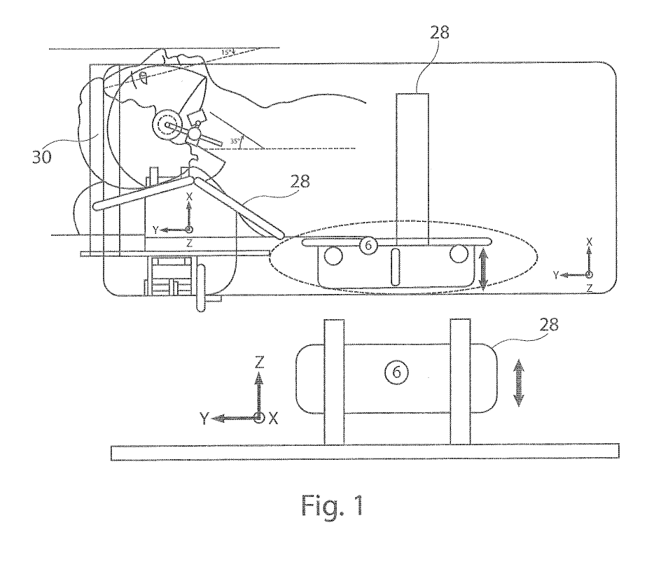

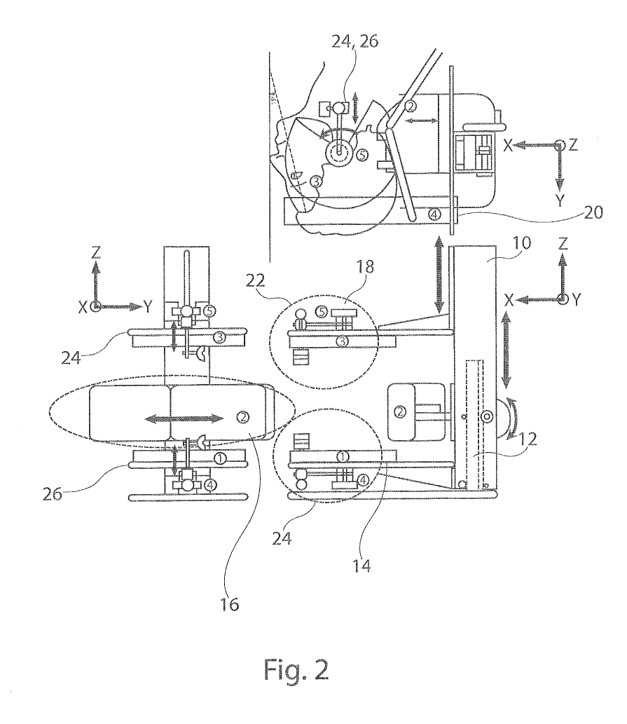

Referring to FIGS. 1 and 2 for the lateral decubitus position, an apparatus and the Steps for implementing the sniff position and jaw thrusts are described below.

Step 1: A carriage subassembly 10 is translated along the Z axis along a base subassembly rail 12 until the support surface 14, is at a comfortable height for a patient lying on his or her left side.

Step 2: A sniff subassembly, surface 16, is adjusted along the Y axis until comfortably aligned with the patient and locked in place.

Step 3: The sniff subassembly, surface 16, is adjusted along the X axis until comfortably aligned with the patient and locked in place.

Step 4: A head clamp 18 is translated along the carriage subassembly along the Z axis until the patient's head is comfortably constrained.

Step 5: A flexible band 20 is placed over the patient's forehead and attached to the back side of the sniff subassembly constraining the patient's head in rotation about the Z axis.

Step 6: The vertical adjustment arms of a left and right jaw clamp subassemblies 22, are moved along the Z axis until aligned with the patient's mandible.

Step 7: Vertical adjustment arms 24, 26 are adjusted radially about the Z axis until in line with engaging the patient's mandible.

Step 8: The left and right jaw claw subassemblies 22, 24 are rotated about the Z axis until the mandible is engaged and extended to the desired amount.

Step 9: A backboard subassembly 28 height is adjusted along the Z axis until aligned with the center of the back.

Step 10: The backboard subassembly 28 position relative to the back is adjusted along the X axis to support maintenance of the patient at a 35° sniff position angle of the head.

Step 11: A flexible band 30 is placed around the abdomen of the patient and the back surface of the back board subassembly 28 to constrain the patient in the X-Y plane.

The present invention as above described provides several distinct advantages. These include:

(1) Achieving a desired position also known as the sniffing position or chin-lift (35° of neck flexion and 15° of head extension) without the use of jaw support members that may cause stimulation, and that is comfortable for the patient while in the lateral decubitus position;

(2) Alignment of 3 axes (oropharyngeal, laryngeal, tracheal) to provide the most optimal view for intubation in the lateral decubitus;

(3) Restrain of the patient's head from moving and disengaging the patient from a desired position;

(4) Provides an easy, user friendly mechanism for the jaw thrust maneuver to be performed in a hands free fashion while the patient lies on either of his/her side;

(5) A durable device with inexpensive disposable parts that may come in contact with the patient;

(6) A device that is easily disinfected;

(7) A device that is MRI or Xray compatible; and

(8) Provides the most amount of exposure to the surgical field.

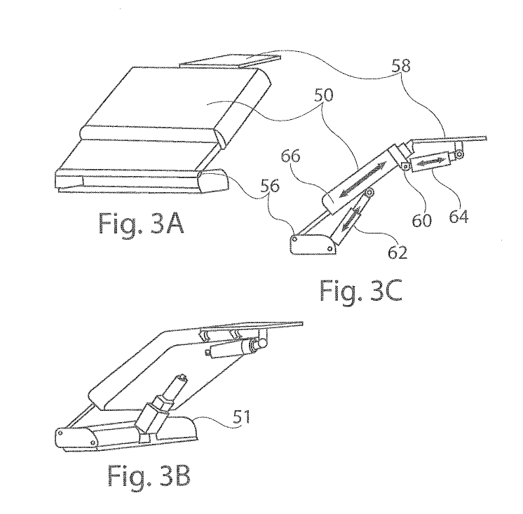

Still yet other embodiments of the invention for the supine position are shown in FIGS. 3-7. Referring next to FIGS. 3A-3C, a patient positioning device is provided which includes:

1. An adjustable ramp 50 that fits the torso of the patient. Ramp 50 includes a base which attaches to the operating room table 54. A pivot axis 56 allows the ramp to rotate relative to the operating table 54 at the base.

2. A lift top 58 that accommodates the neck and head of the patient, and includes a pivot axis 60 that allows the lift top to rotate relative to the ramp

3. Linear actuators that extend or retract along the indicated axes. The linear actuators include a first linear actuator 62 that connects between base 51 attached to operating table 54 and the back of the ramp 50. Ramp 50 is hingedly attached to the base 51. One or more actuators can be used to provide the required force. Actuation results in a change in actuator length L_{A1}. A second linear actuator 64 connects between back of the ramp 50 and the back of the lift top 58, via a hinge 55 between the ramp 50 top and the lift top 58. One or more actuators can be used to provide the required force. Actuation results in a change in actuator length L_{A2}. A third linear actuator 66 attached to ramp 50 is used to extend and retract ramp length to meet a required patent torso length. One or more actuators can be used to provide the required force. Actuation results in a change in actuator length.

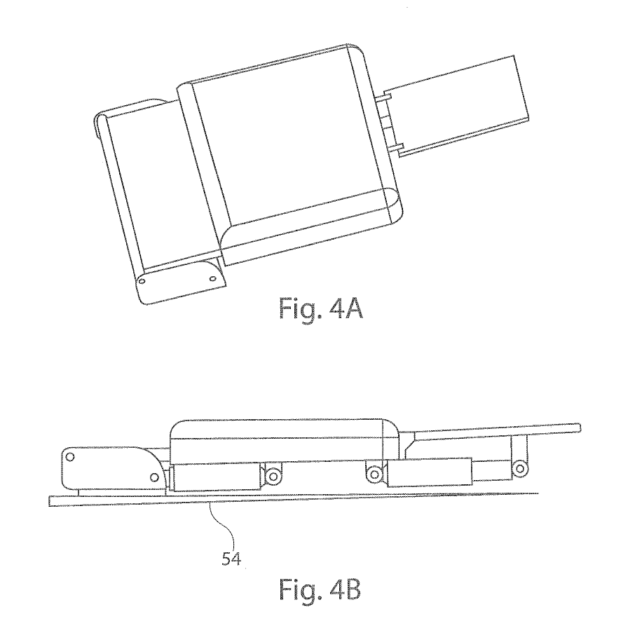

FIGS. 4A-4B show the device of FIGS. 3A-3C in a lowered position.

In yet another embodiment, the positioning device may be incorporated into an operating table, or retrofit to an existing operating table. In this later embodiment:

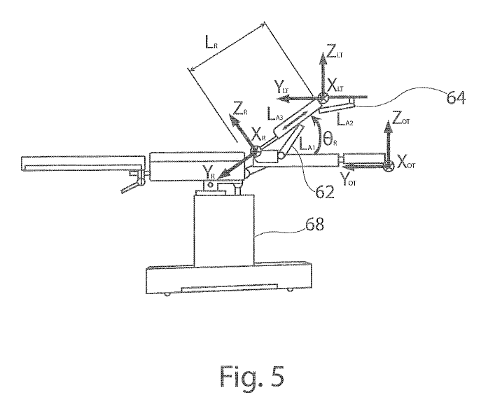

1. The device can retrofit to an existing operating table 68 or be incorporated into the design of a new table as shown in FIG. 5.

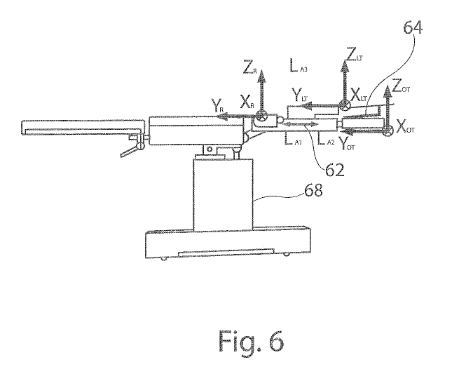

2. The ramp is raised and lowered relative to the operating table through the respective extension or contraction of linear actuator 62. The ramp pivots about the X_{R} axis resulting in a change in θ_{R} as shown in FIGS. 5 and 6.

3. The lift top (LT) is rotated relative to the ramp about the X_{LT} axis as shown in shown in FIG. 5 and FIG. 6 when the linear actuator 64 is extended or retracted.

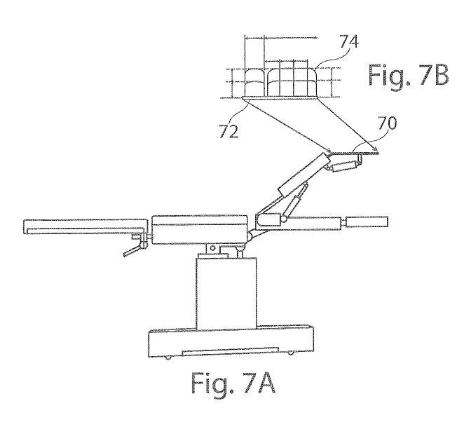

4. The extension of the linear actuator 64 can be operated independent of lift actuator 62 to result in an inclined position of the lift top about the X_{LT} axis. The extension or retraction of linear actuator 64 can also be coordinated with the extension or retraction of linear actuator 62 to maintain the angle of the lift top relative to the operating table constant as the angle θ_{R} is varied due to the change in length of linear actuator 62 as illustrated in FIG. 6 and FIG. 7 where the lift top remains parallel to the top of the operating table.

5. The ramp length, L_{R}, can be controlled by the extension or retraction of linear actuator 62.

6. If desired, a jaw claw as will be described in detail hereinafter can be integrated into the lift top of the system.

Yet another embodiment, illustrated in FIGS. 7A-7B the apparatus includes a lift top 70 which interfaces with a patient's head and neck. Lift top 70 comprises two elements 72, 74 that adjust in the Z direction to interface optimally with the neck and head as shown in FIGS. 7A-7B. The neck and head adjustments are independently adjustable in the Z direction from the nominal location and comprise pneumatically driven pillows or mechanically driven pads. Another option is to have only the neck or head portions adjust and the corresponding head or neck regions be stationary pads. The nominal and extended ranges for each are illustrated.

The present invention also addresses problems encountered with the use of a face mask, including maintaining the face mask on the face of the patient during a procedure, and especially during a long term respiratory event. Also, patient movement can cause a face mask to fall off, as can incidental contact.

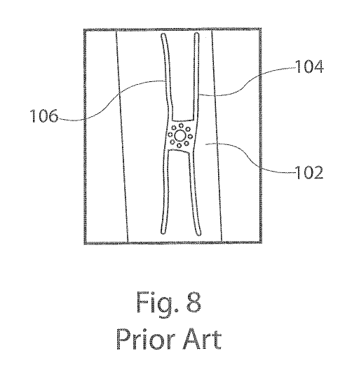

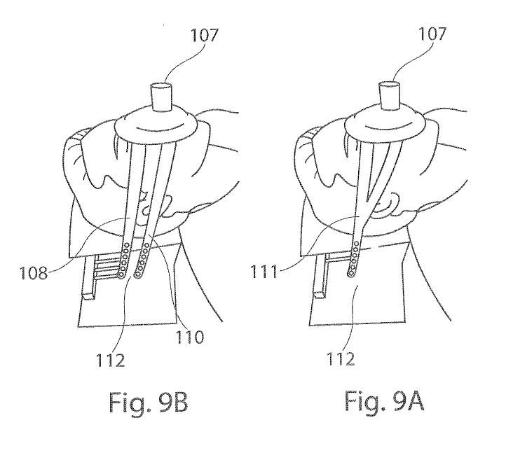

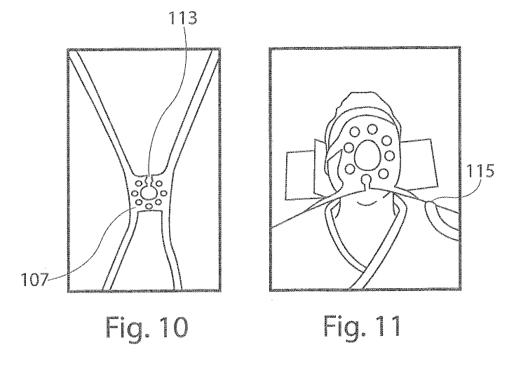

Referring to FIG. 8, a conventional patient mask strap 102 is illustrated. The mask strap 102 comprises one or two straps 104, 106 which are designed to be tied to the back of the patient's head. Referring to FIGS. 9A-9B, the present invention provides a mask 107 in which straps 108, 110 (FIG. 9A) or conjoined straps 111 (FIG. 9B) are placed over the mask and anchored to a base plate 112 under the patient's head. In a case where the ventilation hose has already been attached to the mask, the mask 107 may be split at one side 113 to accommodate the ventilation hose 115, and still allow the attachment of the mask to the base as illustrated in FIGS. 10 and 11.

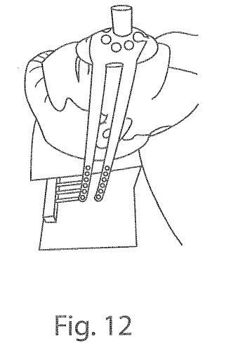

In a situation where the oxygen hose has yet to be attached to the mask, an unseparated mask strap can be attached to the mask, then to the hose, then to the patient as illustrated in FIG. 12.

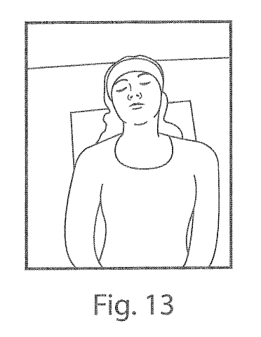

In addition to strapping a patient and mask to the base, other parts of the patients head can be attached to the base if the head needs to be constrained as illustrated in FIG. 13.

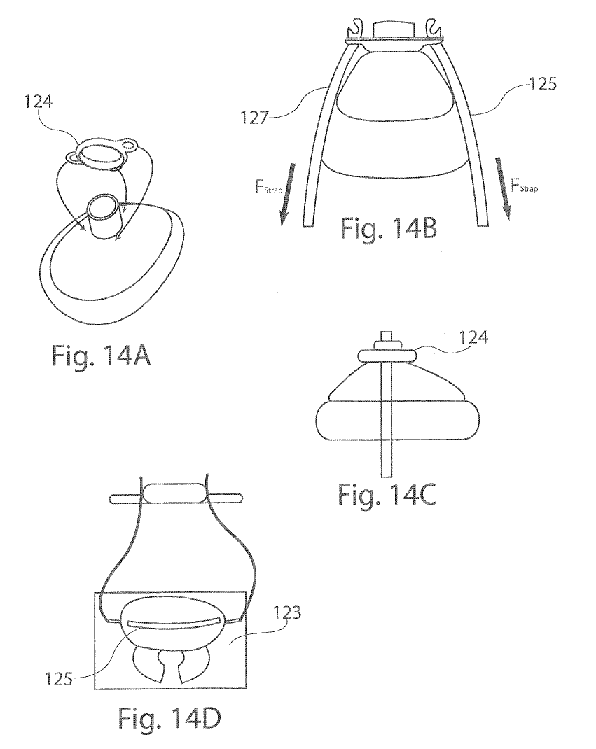

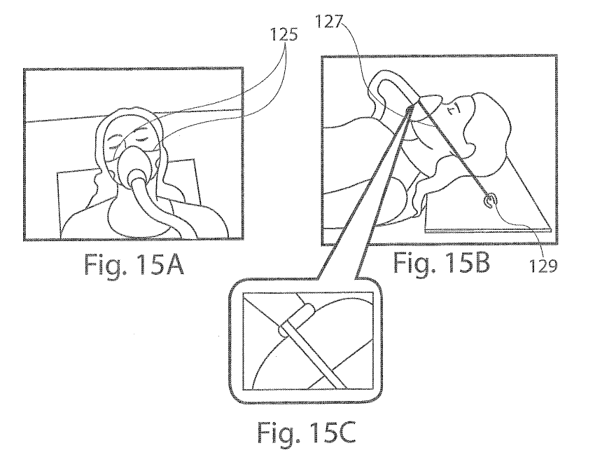

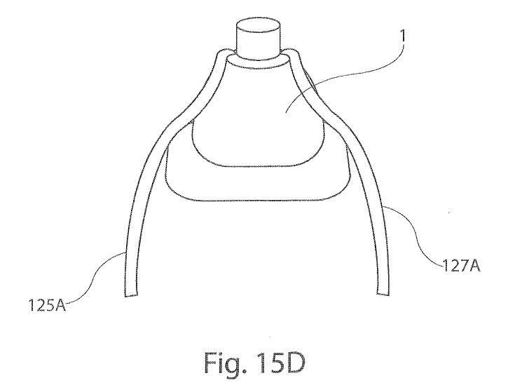

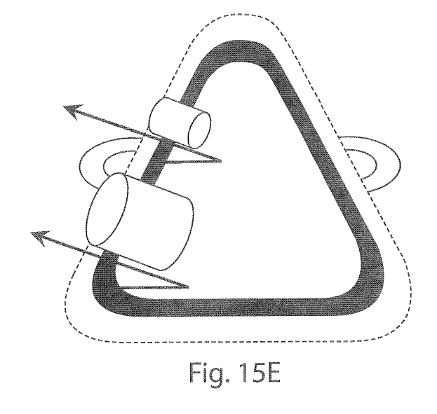

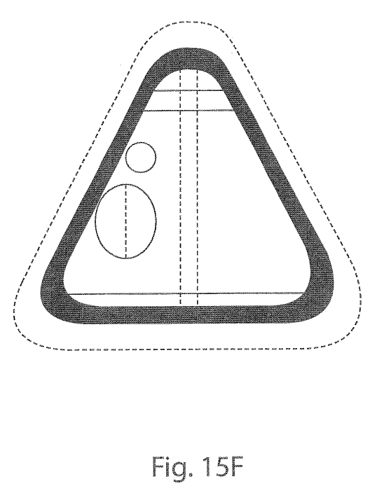

FIGS. 14A-14D illustrate yet another embodiment of our invention, in which the ventilation mask 120 is attached with a strap 122 from the front to a base such as a patient support, where the strap consists of elastomeric straps that may vary in diameter of from, e.g., 0.125″ to 0.25″. A mask anchor ring 124 kinematically interfaces with the mask at a plurality of interface points, preferably three, on the mask anchor ring, resulting in more evenly applied force to the mask, as shown in FIGS. 15A-15C or the mask can have the mask anchor straps (one or more on each side of the mask) built into the mask, where the mask anchor ring would not be needed (see FIG. 15D). Preferably one or more elastomeric straps 125, 127 are affixed to a mask anchor ring 124 or built into the mask (FIG. 15D, straps 125A, 127A), spaced at any number of degrees apart an imaginary circle, for example if four straps where used then they would be spaced 90° apart, where each strap would secure each of the four sides of the mask (the right side, left side, forehead side, chin side). The mask is attached over the nose only or the nose and mouth of the patient by a force, F_{Strap} applied by the elongated straps that connect to a head support.

A single mask anchor strap 125, 127 on each side configuration is shown in FIGS. 15A-15C. Each mask anchor strap attaches posteriorly behind the head to a respective mask anchor clip 129, 131 attached to the head support with a friction connection. Alternatively, the straps may include a plurality of holes for attachment to prongs on the head support. This connection results in an essentially airtight seal between the ventilation mask and the patients face.

An advantage of the mask strap of the current invention over conventional masks is that it allows a doctor to approach a patient from in front of the patient's face, place either the nasal mask, full face mask, or combined nasal-oral mask and then attach either of the masks to the surface so that the patient's head is resting on a surface and it prevents the patient's head from moving out of the desired position. Thus, if the patient's head is already on the surface, the provider will not have to lift the patient's head in order to strap the mask to the patient's face. Also, this approach places the patient in the desired position and fixes their head and neck in this position to maintain a patent airway. The mask strap of the current invention is smaller than a conventional mask and only comprises two surfaces of which an aperture is in the center that is placed on the face mask and two arm extensions with a plurality of holes that connect to a surface. It does not contain a wide rectangular head rest that wraps around the patient's head as in the case of conventional masks. It also does not require prongs on the face mask, and thus eliminates the risk of injury to the provider and patient.

Another advantage of the present invention is that it both maintains the sniffing position by fixing the patient's head to the table and is placed in front of the patient's face. Therefore if the patient lies down the strap can be applied without having to lift the patient's head off of the table.

In another aspect of the invention there is provided either a disposable or re-usable nasal mask with an off-centered aperture for ventilation and/or one for Oxygen or a combined but detachable and either disposable or re-usable nasal mask and oral mask, which can be used either uniformly as a full facemask to ventilate a patient either prior to endotracheal intubation or during general anesthesia (GA), or the mouth mask can be separated from the nasal mask and the nasal mask can be used to apply nasal non-invasive positive pressure ventilation (BiPAP—Bilevel Positive Airway Pressure/CPAP—continuous positive airway pressure) to help maintain a patent airway and ventilate a patient while the anesthesiologist is attempting intubation, which will significantly prolong the time until the patient begins to desaturate. More specifically, the present invention also provides a facemask, which is capable of functioning as an improved anesthesia mask compared to the prior art masks because it uniquely combines the following advantages:

(1) the ability to deliver and evacuate gas(es) while being sealed on the patient's face,

(2) the provision of either a full face mask having a separate nasal mask or a nasal mask alone to apply nasal non-invasive positive pressure ventilation (BiPAP/CPAP) and/or oxygenation during apneic periods (time when patient is not breathing on their own), sedation cases, general anesthesia (GA), and for respiratory therapy, and it has a separate mouth mask, which when attached to the nasal mask is essentially a traditional full facemask used for oxygenation and ventilation during bag-mask ventilation, GA, and respiratory treatments, or the mouth mask can be detached in order to provide the anesthesiologists with access to the airway for intubation and fiberoptic intubation,

(3) secure the nasal mask and nasal-oral mask not only to the patient's face but also secure the patient's head and neck in position to maintain airway patency, and stabilize the mask on the patient's face without affecting its sealing capability, and

(4) detach the oral mask or use the stand alone nasal mask and attach the nasal mask and use the head rest and/or neck rest cover with the mask strap to clip onto the nasal masks from the front and secure the nasal masks to the patient's face and attach the nasal mask to a portable oxygen supply source and use the nasal mask to supply oxygen during patient transport.

(5) another advantage is that the off-center port or ports will minimize the obstruction of the anesthesiologist's glottic view during the intubation process. On-center ports will partially or completely obstruct the glottic view.

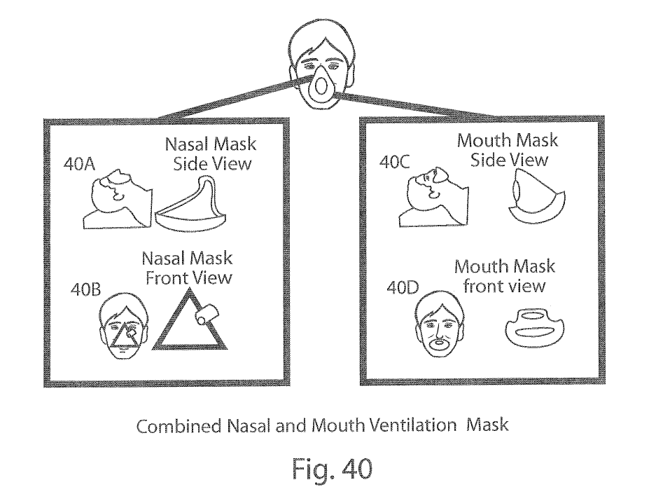

Referring to FIGS. 40A-40B, the top left picture shows a side view of the nasal mask and the bottom left picture shows a front view, which consists of three surfaces; where the first surface is the bottom surface, is open, with a soft, flexible, pneumatic, border that contours to nose bridge, side of the nose, cheeks, and upper lip in order to create seal when in contact with the patient's face. The bottom surface also has a plurality of holes on each side, which allow straps to be either attached to or detachable and used to secure the mask to the patient's face and the patient's head and/or neck in the desired position. Built into these plurality of holes are clips which allow a mask strap to attach to when the mask strap comes from behind the patient's head and attaches to these clips in front. The second surface is the top surface of the nasal mask and contains one or more openings, the first of, which can be either off-centered left or right and connects to either an anesthesia circuit, BiPAP/CPAP machine, or resuscitation bag, in order to prevent obstruction of the glottic view or it can be centered and connects to the breathing circuit to enable gas exchange. A second opening can be connected to either an End-tidal CO2 monitor or a portable oxygen supply. A third opening can be used to allow for the use of both supplemental oxygen from an oxygen supply device and used to monitor end-tidal CO2. The third surface is base surface which may or may not consist of an opening as well as a door, which when the door is opened by engagement of the mouth mask it will allow for bilateral transfer of gases and when it is closed by disengagement of the mouth mask it will prevent gases from escaping from the nasal mask.

Referring to FIGS. 40C-40D, the top right picture shows a side view of the oral mask and the bottom right picture shows a front view of the oral mask, which also consists of three surfaces; where the first surface is the bottom surface, is open, with a soft, flexible, pneumatic, border that contours to lower lips, cheeks, and upper lips in order to create seal when in contact with the patient's face. The second surface is the top surface. The third surface is the base, which consists of a connector, which when engaged with the nasal mask door, pushes the door open and allows for the bilateral flow of gases and when the connector disengages, it causes the door of the nasal mask to close shut in order to prevent leakage of gases.

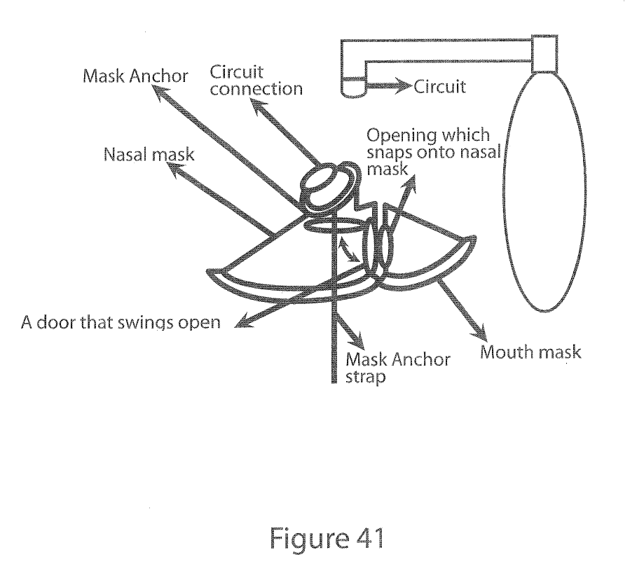

FIG. 41 shows the oral mask connector within the mouth mask engaging with the nasal mask, which causes the nasal mask door to swing open and allow gases to flow into both the mouth and nose bilaterally. When the mouth mask is engaged with the nasal mask, the combination creates the traditional facemask, which can be used for bag-mask ventilation, general anesthesia, respiratory treatment, etc. When the oral mask connector disengages from the nasal mask, the nasal mask door closes, which prevents gases from escaping and now turns the traditional facemask into a nasal mask which can be used for nasal BiPAP/CPAP for sedation cases, ventilation during intubation, general anesthesia, respiratory treatments, can be attached to a portable oxygen supply source and used to deliver oxygen for patient transport, etc. FIG. 41 also shows the mask anchor, which can either surround the opening of the nasal mask or be built into the nasal mask (FIG. 41A) and attaches to a surface, which secures the nasal mask and/or the nasal-oral mask to the patient's face in order to keep a tight seal, prevent leakage of anesthetic gases, and also maintains the patient's head and neck in the desired position to ensure an open airway.

While the invention has been described for use in connection with surgery, the invention also may be used during sedation cases, especially deep sedation or patients with Obstructed Sleep Apnea (OSA) or obesity, where the upper airway of many of these patients becomes obstructed and prevents them from breathing. The oral mask of the current invention can be separated from the nasal mask and the nasal mask or just the stand alone nasal mask can be used to apply BiPAP and/or continuous positive airway pressure (CPAP) to help relieve the upper airway obstruction, maintain a patent airway, and assist in ventilation during the case. Another advantage of the current invention as opposed to existing nasal masks is in situations where a nasal mask is not sufficient to ventilate the patient. The proposed invention has the ability to reattach the oral mask and now the mask can be used for traditional bag-mask ventilation. Another advantage of the invention is the ability to apply nasal BiPAP/CPAP during semi-awake fiberoptic intubations, where being able to maintain a patient's oxygen saturation levels are also critical. Another advantage of the current nasal mask and/or nasal-oral mask is the ability to connect it to a portable oxygen supply of needed and used to transport the patient with oxygen. It can also attach to both a supplement oxygen supply source as well as a resuscitator bag simultaneously in order to provide simultaneous oxygenation and ventilation. The final advantage that the present invention has over the prior anesthesia mask art is the ability to secure not only the combined nasal mask and oral mask to the patient's face allowing for hands-free ventilation, but it also secures the patient's head and neck in place by attaching to a surface and maintaining the patient in a position that ensures a patent airway, which is critical for oxygenation and ventilation.

In yet another aspect, the present invention provides improvement over the system described in our aforesaid parent applications, by providing a simple and elegant head/neck rest that comprises a compliant adjustable head and neck rest (ie: bellows, mechanical) that is independently controllable to support a patient's head/neck to obtain an optimal sniff position by raising a patient's neck and head independently of the carriage.

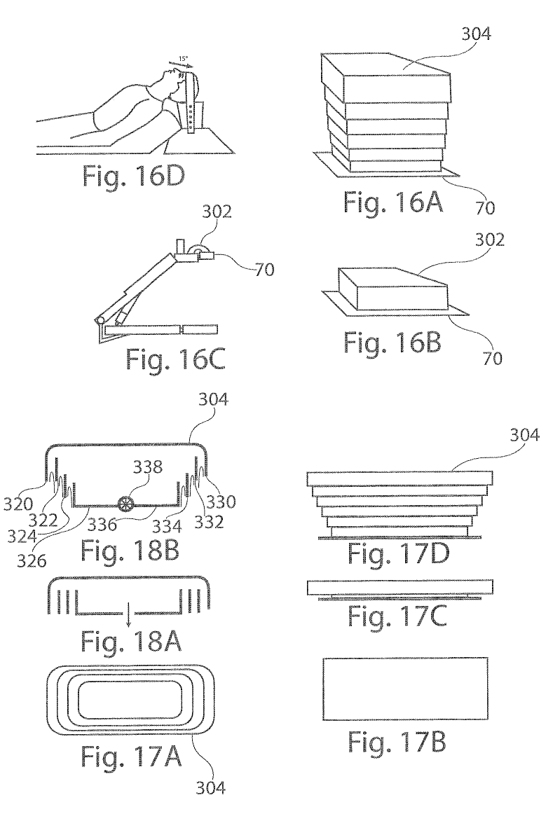

Referring to FIGS. 16-18, another feature and advantage of the present invention is to provide a head/neck support 302 that may be independently controlled to support the optimum sniff position by raising a patient's head and neck independently of the ramp, whereby to improve the glottic view in real time. More particularly, in accordance with the present invention, there is provided an adjustable head/neck support mechanism that permits adjustment in the z-axis. In one embodiment the adjustable head/neck support mechanism consists of a pneumatic jack such as a compliant bellows 304 that is fixed or located on top surface of lift top 70. Alternatively, as will be described below the adjustable head/neck support may comprise a mechanical jack.

Referring again to FIGS. 16-18, pneumatic head/neck support 302 in accordance with the present invention comprises an inflatable bladder or bellows 304 comprising a plurality of rigid concentric rings 320, 322, 324, 326 joined by flexible membranes 330, 332, 334 on a rigid base 336. The bellows includes a two-way valve 338 through which air may be added or subtracted in order to inflate the bellows and increase internal pressure and bellows height. The relative (height) position of a patient's head or neck, and achievement of an optimal sniff position, may be controlled by regulating the pressure within the bellows. Thus, when air is flowed into the bellows, the head/neck bellows 304 raises, and when air flows out of the bellows, the head/neck bellows 304 lowers. A feature and advantage of the present invention is that the inflatable bellows provide a relatively low-cost highly-adjustable device that readily may be sterilized. However, the device also is sufficiently low cost that it could be used once, and then discarded. Moreover, lateral movement of the head/neck rest is significantly more constrained than if the head/neck rest were made of a purely compliant bellows for raising and or lowering the head/neck.

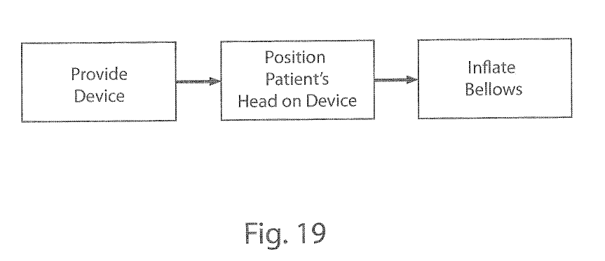

Referring to FIG. 19, the present invention also provides methods for positioning a patient. The methods include the steps of: providing a device according to the invention as described herein, placing the patient's head substantially on the top of the adjustable mechanism (bellows), elevating the ramp and then adjusting the mechanism so that the patient is in a sniff position.

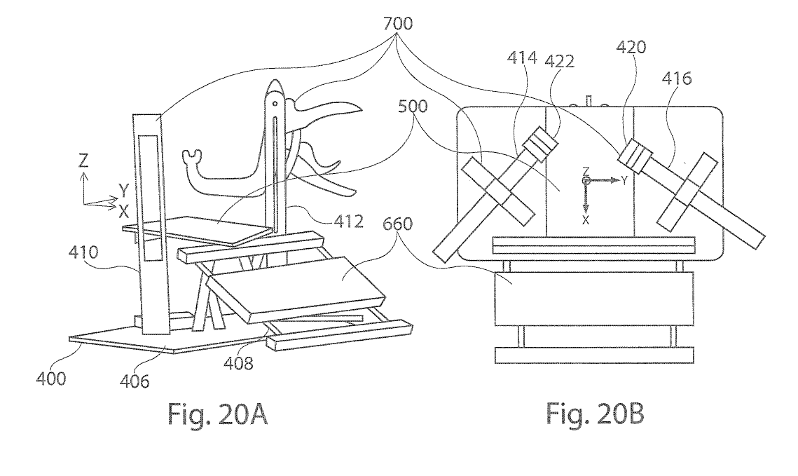

Referring in particular to FIGS. 20A and 20B of the drawings, in another aspect, the present invention provides a device comprising:

a base 400 comprising a first side which supports the patients head and neck, a second side acting as the foundation, inner vertically adjustable support structures 406 between the first and second sides, and a detachable third inclined side or ramp 408 which supports the upper back, middle back, and shoulders of a patient and is rotatable and adjustable in the x axis;

a first support 410 positioned on the second side of the base and lockably adjustable with respect to the second side of the base in an x and y axes;

a second support 412 positioned on the second side of the base and lockably adjustable with respect to the second side of the base in the x and y axes;

a first mandible arm 414 extending from a first vertically adjusted portion of the first support 410, wherein the first vertically adjusted portion is lockable in a z axis to lockably adjust the first mandible arm with respect to the z axis, and wherein the first mandible arm 414 is positionable to be in contact with the patient's jaw; and

a second mandible arm 416 extending from a second vertically adjusted portion of the second support 412, wherein the second vertically adjusted portion is lockable in the z axis to adjust the second mandible arm 416 with respect to the z axis, and wherein the second mandible arm 416 is positionable to be in contact with the patient's jaw;

wherein the first mandible arm 414 and the second mandible arm 416 are movable such that each is positionable to be in contact with the patient's jaw and to maintain the patient in a desired position. In such case, preferably the sniffing position, aligning all 3 axes (oropharyngeal, laryngeal, tracheal), and/or in the jaw thrust maneuver, and leaving the provider hands free.

In a preferred embodiment, the base 400 is rectangular.

In one embodiment, the mandible arms 414, 416 are positionable to be in contact with the patient's jaw at three points. The most distal end of the mandible arms can pivot in three degrees in order to conform to the angle of the mandible.

The first mandible arm 414 and second mandible arm 416 each include a mandible pad 420, 422 which preferably are formed of a resiliently deformable material such as foam. In yet another embodiment, the first mandible arm 414 and second mandible arm 416 are removeably connected to the first support 410 and second support 412, respectively; and the most distal end of the first and second mandible arms 414, 416 are removeably connected to the distal end of the first and second supports 410, 412.

In yet another embodiment, the first mandible arm 414 is moveable relative to the first support 410, and the second mandible arm 416 is moveable to the second support 412.

In further embodiments, the mandible arms 414, 416 are positionable such that the mandible pads 420, 422 are in contact with the patient's jaw at one or more points.

In some embodiments, the mandible arms 414, 416 are positionable such that the mandible pads 420, 422 are in contact with the patient's jaw at three points, and in yet other embodiments the first support 410 is moveable relative to the base 400 and the second support 412 is moveable relative to the base 400.

In various embodiments the first support 410 is moveable relative to the base 400 on the X and Y axis, and the second support 412 is movable relative to the base 400 on the X and Y axis.

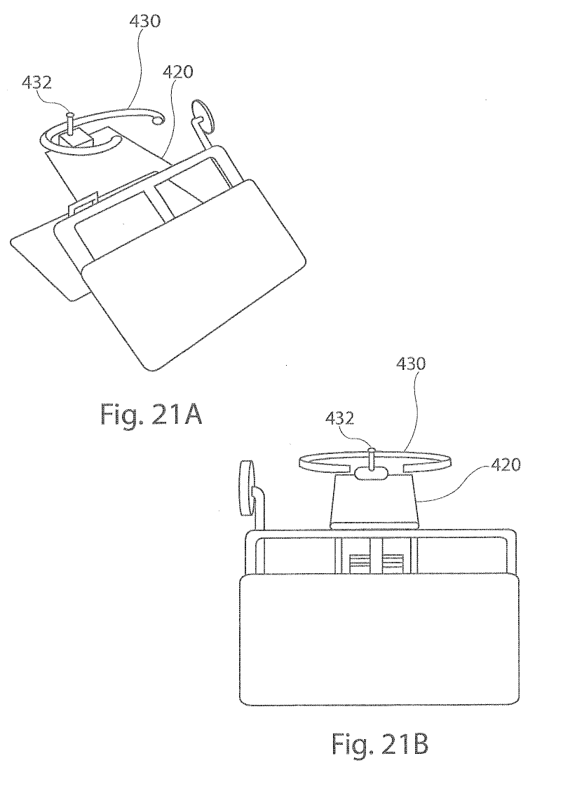

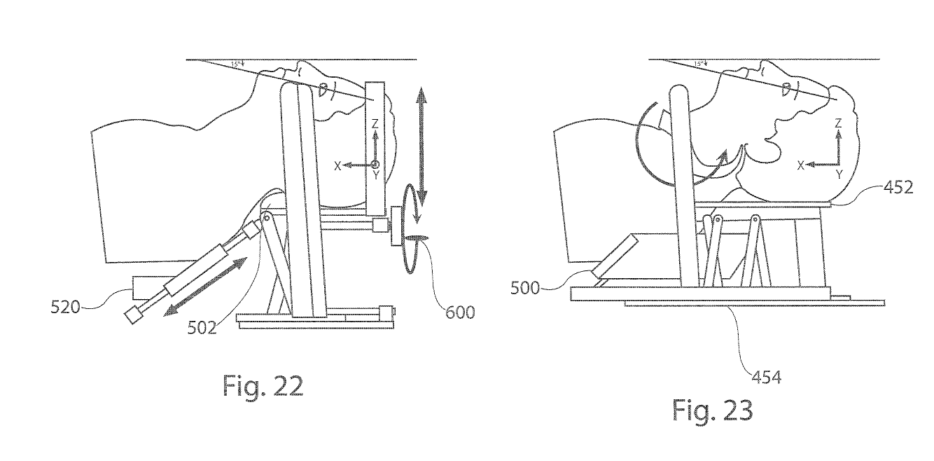

Referring in particular to FIGS. 21A and 21B, in one embodiment of the present invention, the mandible arms each include a curved portion 430 that consists of an adjustable and lockable mechanism 432 that attach to a respective mandible pad 420, 422, wherein the mandible pads 420, 422 are flexible, and wherein the mandible pads have a distal side configured to attach to the curved portion and a proximal side configured to contact a patient's jaw at a plurality of points, which can pivot in all angles at the distal end; and a connector portion which is configured to attach to a support.

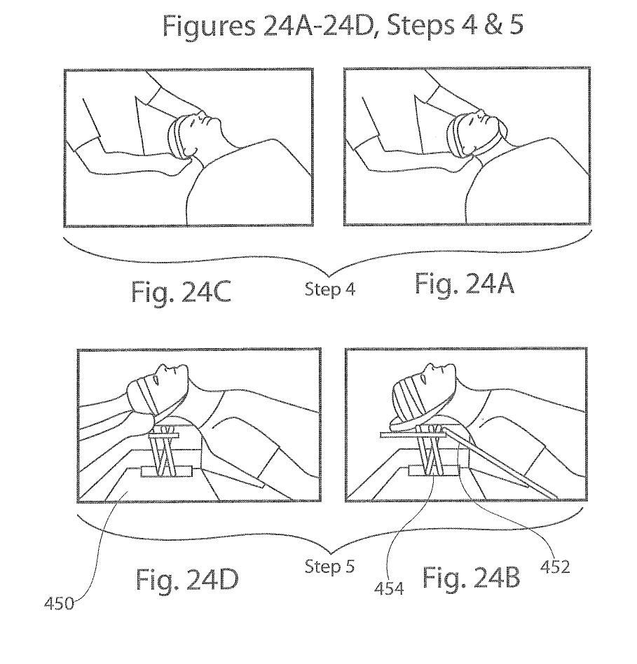

Referring to FIGS. 22-24, in yet other embodiment, a connector portion 450, is configured to attach to a support 452 that is attached to a base 454 comprising a left side and a right side, wherein the base 454 is configured to substantially accommodate a patient's neck and head, and wherein the support 452 is moveable in three axis such that the mandible pads 420, 422 are positionable to be in contact with the patient's jaw at one or more points and to maintain a desired position.

A further embodiment to the present invention includes a detachable inclined surface 500 that may attach to the base 454, and the angle at which the inclined surface 500 is positioned can be changed, while also having the ability of extending further distance in order to maintain the same angle which assists in supporting larger patients. The inclined surface will have a back and shoulder bar 502 that rests on it which will support a patient's upper back, middle back, and shoulders. This support will enable gravity to displace weight off of patient's chest, allowing for a more patent airway.

In yet another embodiment of the present invention, there optionally is included a measuring device 530 such as a level to confirm the optimal neck flexion angle of 35°. One embodiment consists of two sides, the first side 532 of which is semi cylindrical and consists of four arms 554, 556, 558, 560, each of which is located within each of the four corners, each of which makes contact with the patient's neck; the second side 562 which consists of a leveled angle.

In yet another embodiment the present invention optionally includes a leveling device 564 used to confirm the optimal head extension angle of 15°. This latter embodiment consists of two sides, the first side 566 or triangular side which consists of three arms 568, 570, 572, each of which is located within each of the three corners, each of which is adjustable in the z-axis, each of which makes contact with the patient's face; and the second side 574 which consists of a 15° leveled angle.

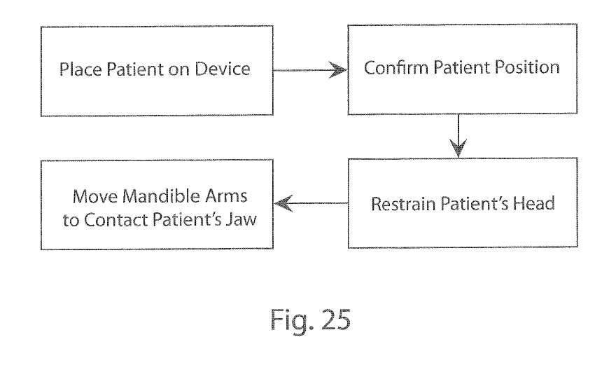

Referring now to FIG. 25, in yet another embodiment, the present invention includes a method for positioning a patient including the steps of: placing the patient's upper back, middle back, and shoulders on an inclined surface along with the patient's neck and head substantially on the base of the device; placing the patient's head and neck in the desired position, confirming the position with a measuring device; restraining the patient's head to prevent the patient from being dislodged from the desired position; moving a first mandible arm to contact the patient's jaw; moving the second mandible arm to contact the patient's jaw; wherein the contact of the first mandible arm and the second mandible arm provides sufficient force to substantially maintain the patient's head, neck, and/or jaw in a desired position.

The invention will be further illustrated with reference to FIGS. 22-26 which illustrate the Steps for implementing the Sniff position and Jaw thrusts using the above described device as follows:

Step 1: Adjust Head Height in z with Jack Subassembly to Achieve 35° Angle by rotating handle 600 and engaging the acme screw.

Step 2: Adjust Ramp Length.

Step 3: With correct radius Head Rest in place, constrain forehead to Jack Subassembly with flexible band to achieve 15° angle.

Step 4: Attach the Jaw-Claw-Based Subassembly to the top portion of the Jack Subassembly as illustrated in FIG. 4 with the Claws separated (by rotating opposite one another along the Z axis) and tilted below the plane of the head (Rotated about the Y axis). Nominally position the left and right padded elements of the Claw below their respective mandible locations (by rotating them towards each other about the Z axis) and slide the Claw assembly along the X axis and lock the Claw assembly in position to the top of the Jack subassembly. Adjust the Left and Right Jaw Thrust Subassemblies by sliding them along the y axis along the Jaw Thrust Slide towards the patient's head and squeezing the Jaw Thrust Grip causing it to rotate about the x axis, resulting in a translation in the positive z direction until the Jaw Thrust Shelf is lightly engaged below the mandible, and locked into position.

Step 5: With the claw subassembly locked in place on the Jack Subassembly, Rotate, about the Z axis, the left and right arms of the Claw subassembly until the padded portions are engaged with their respective left and right mandible of the jaw. After the pads are securely engaged with the mandible, rotate both left and right arms of the claw about the −Y direction of the Y axis until the Jaw is extended by the desired amount in the Z direction. Extend Mandible by required amount by squeezing the Left and Right Jaw Claw Grips simultaneously resulting in a rotation of the Jaw Claw and engagement with an extension of the mandible.

Lock the Left and Right Jaw Thrust Assemblies to the Jaw Thrust Slide by rotating the Jaw Thrust Lock about the x axis. This fixes the Jaw Thrust Assembly position in the x-y Plane. Proceed to engage the Left and Right Jaw thrust shelf to extend the mandible by the required amount of squeezing the Jaw Thrust Grip, causing it to rotate about the x axis, resulting in a translation in the positive z direction until the Jaw is properly extended and the Airway is open.

The foregoing steps:

(1) achieve a desired position also known as the sniffing position or chin-lift (35° of neck flexion and 15° of head extension) without the use of jaw support members that may cause stimulation and that is comfortable for the patient;

(2) Restrains the patient's head from moving and disengaging the patient from the desired position;

(3) Achieves a position in obese patients where the ear and the sternum are aligned in horizontally to achieve maximal air exchange;

(4) Provides an easy, user friendly mechanism for the jaw thrust maneuver to be performed in a hands free fashion;

(5) Displaces weight off of a patient's chest with the help of gravity by using an inclined surface;

(6) Provides a mechanism of elevating and lowering obese patients without the use of health care workers to limit work related injuries;

(7) Provides a measuring device to confirm the optimal angles of the desired position;

(8) Aligns 3 axes (oropharyngeal, laryngeal, tracheal) to provide the most optimal view for intubation; and

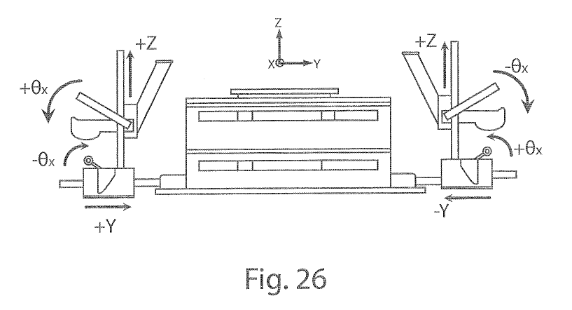

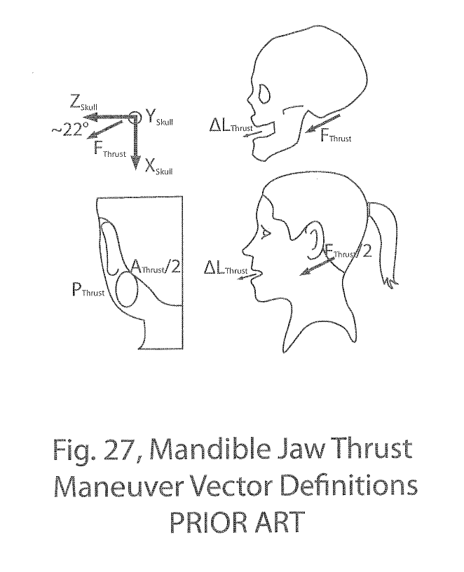

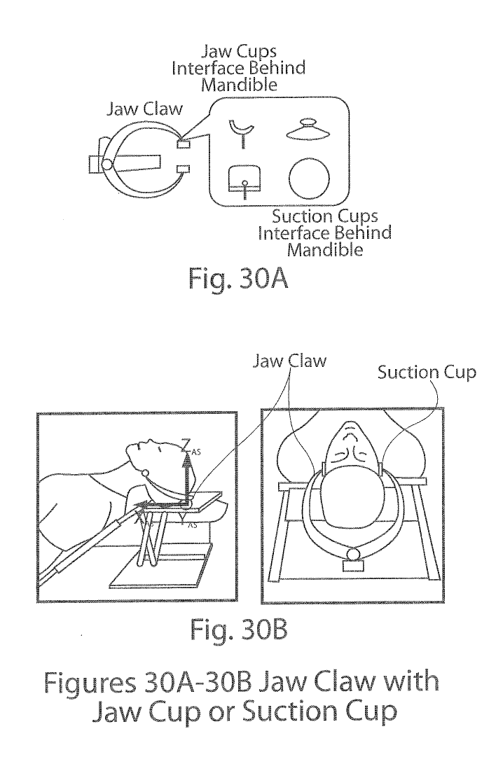

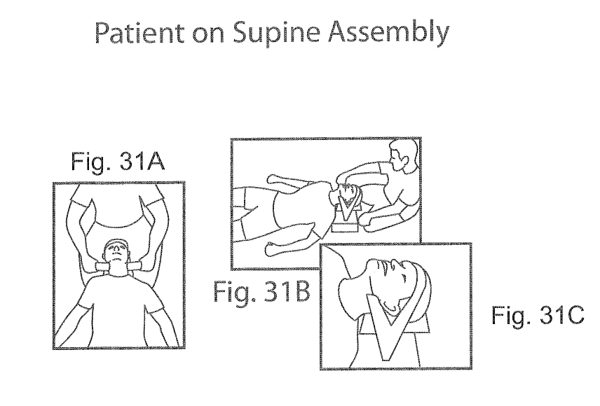

The present invention is believed to be first of its kind to maximize ventilation in a hands free fashion by being able to perform a jaw thrust maneuver, along with the ability to displace weight off of a patient's chest by utilizing an elevating device. The invention also is believed to be the first to utilize a measuring device (i.e.: a level) on the body in order to reassure the provider that the patients head and neck are properly aligned in the sniffing position. This invention also gives the provider the ability to first try a much lesser invasive way of maintaining a patent airway by placing the patient in the proper sniffing position without eliciting any painful stimuli. The provider can then restrain the patient's head to prevent the patient from being displaced from the desired position. If the patient's airway is not yet patent, the provider can use 2 clamps with ratchet capability, each applied to each side of the mandible to displace the jaw forward. If the patient still cannot maintain a patent airway, this invention, by placing the patient in the desired position, aligns all 3 axes in order to achieve the best possible view to intubate the patient. See FIGS. 27-31.

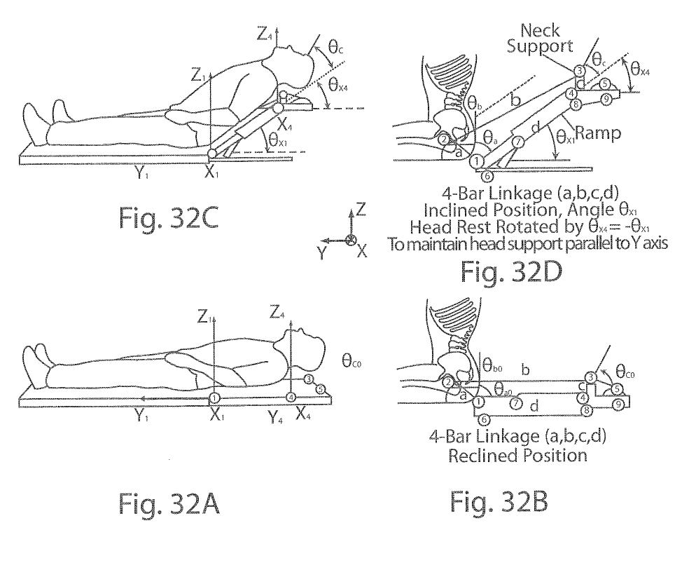

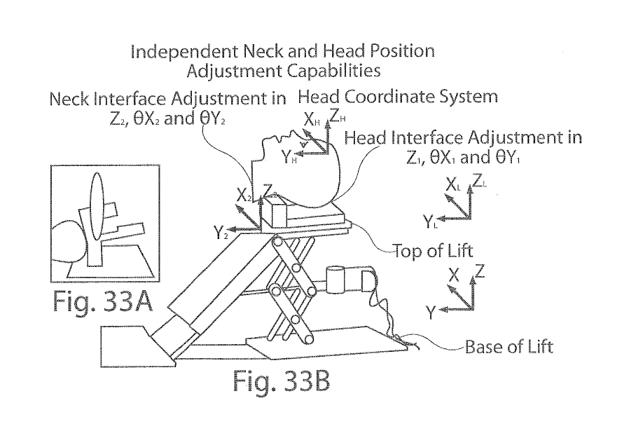

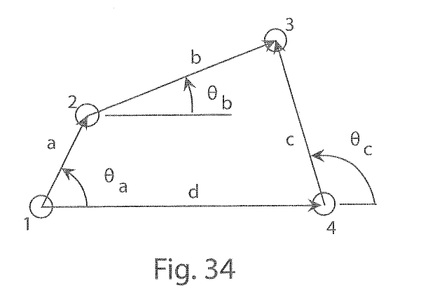

Yet another embodiment of the invention is shown in FIGS. 32A-32D, and 33A and 33B which show the positioning device of the present invention with a patient initially horizontal on the OR table, and raised to an inclined position. When the device is raised and lowered, it pivots about the X axis, Point 1, by an amount θ_{X1}. As the device is raised or lowered, the head support, containing the independently adjustable head and neck actuators that position the head and neck for optimal intubation view, is maintained nominally parallel to the Y axis by rotating about point 4 along the X axis an amount θ_{Z4}, where θ_{X4}=−θ_{X1}. The patient is intubated while in the inclined position and then repositioned to the reclined position for the operation. The device and patient create a 4-bar linkage (FIG. 34) as defined in Table 1 below.

TABLE 1

| Linkage | Linkage Description | Comment |
|---|---|---|
| a | Connects Ramp Pivot Point 1, to Patients | Fixed Length, a |
| | Hip Pivot (Trochanterion), Point 2 | |
| b | Connects Hip Pivot, Point 2, to Top of Neck | Fixed Length, b |
| | actuator (Head/Neck Pivot location), Point 3 | |
| c | Connects Head/Neck Pivot location, Point 3, | Fixed Length, c |
| | to Head Support Pivot, Point 4 | |
| d | Connects Head Support Pivot, Point 4, to | Adjustable |
| | Ramp Pivot, Point 1 | Length, d |

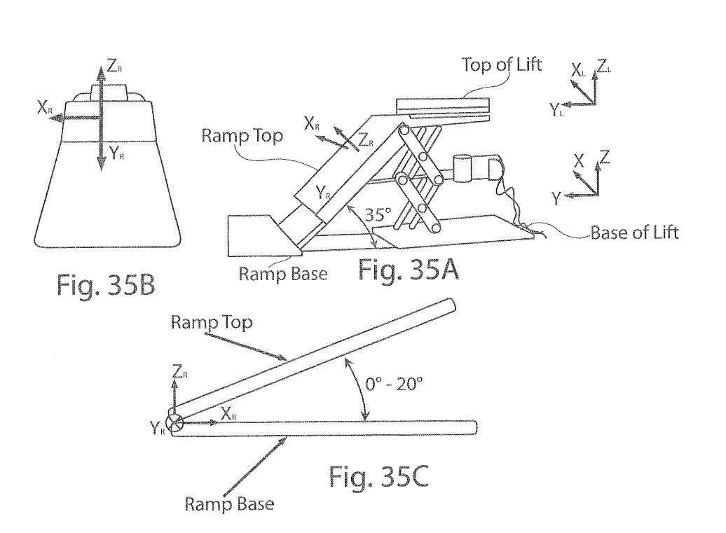

Generalized geometry for the lifting linkage is illustrated in FIGS. 35A-37, and the associated known variables as well as equations for θ₂ as a function of ramp angle θ_{X1}, θ₄ as a function of head support angle θ_{X4} and unknown variables θ₃ and d are defined as follows:
a, b, c, θ′₂, and θ′₄ are known
1. θ₂=θ′₂−θ_{X1} where θ′₂ is for the reclined position and θ_{X1} is the ramp pivot angle about point 1
2. θ₄=θ′₄+θ_{X4} where θ′₄ is for the reclined position and θ_{X4} is the head support pivot angle about point 2
3. θ₃=a sin [(c sin θ₄−a sin θ₂)]/b
4. d=a cos θ₂+b cos θ₃−c cos θ₄

The problem is that as the ramp is raised and, or the head support is rotated about its pivot axes, one or more of the linkage lengths must change or the patient will be forced to slide along the device surface and operating table. The sliding effectively changes linkage lengths a and c, given lengths b and d are fixed. What is desired is to maintain the lengths and positions of linkage a, θₐ and c, θ_{c} fixed relative to their support surfaces as the ramp angle, θ_{X1}, and head support angle, θ_{X4}, are adjusted. The present invention accomplishes this requirement by changing the length of the ramp, linkage d, as a function of the initial patient geometry, ramp angle θ_{X1} and head rest angle θ_{X4}.

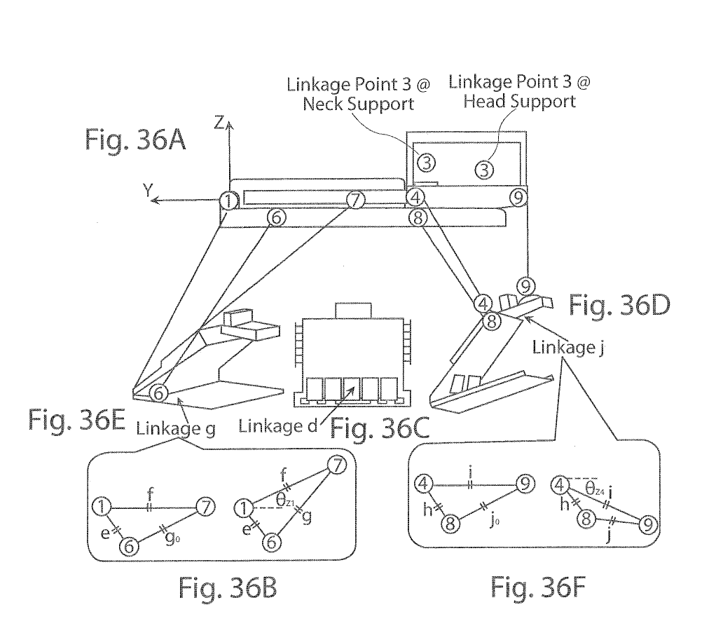

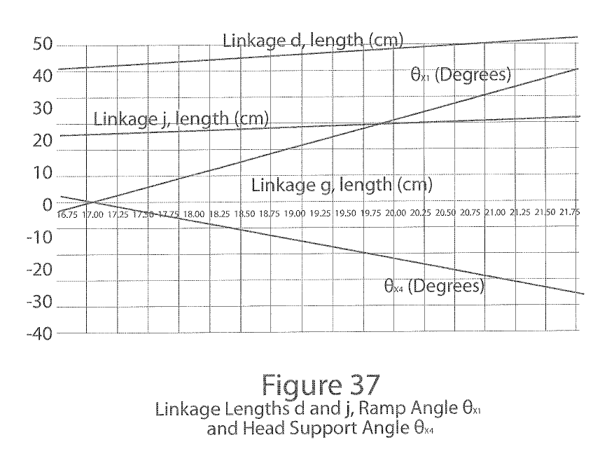

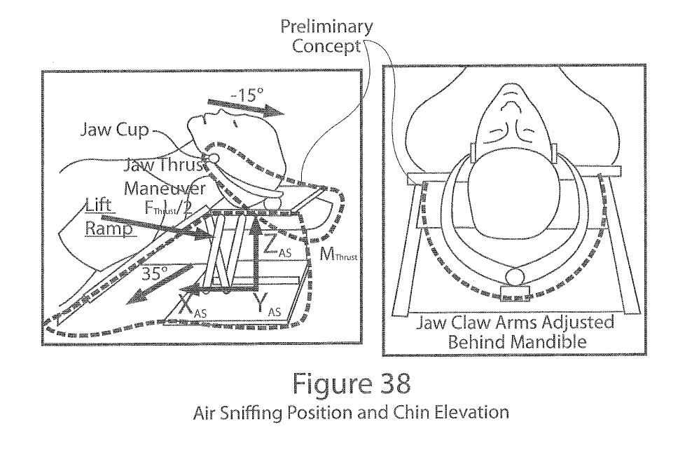

Referring again to FIGS. 36A-36F and 37, as ramp incline is varied, θ_{X1}, the head rest orientation remains parallel, in most cases horizontal to the operating table, by changing head rest angle, θ_{X4} by the opposite amount. This can be accomplished in the following manner:

a. Open loop based on known or estimated geometries of all parameters; or
b. Closed loop where the current angle
θ_{X4} is measured relative to an initial angle, θ′_{X4} and driven back to that initial angel. This could be accomplished with multiple feedback sensors including but not limited to:

i. Measurement of angle relative to gravity with an inclinometer

ii. Encoder

Referring in particular to FIG. 37, as ramp incline is varied, θ_{z1}, and/or head rest angle, θ_{z4} are changed to position the patient, ramp (linkage d) length is varied in order to satisfy the conditions that positions of linkage a, θₐ and c, θ_{c} fixed relative to their support surfaces. This prevents the patient from sliding along the operating table surface as the ramp and head rest angles are adjusted. This can be accomplished in the following manner:

a. Open loop based on known or estimated geometries of all parameters

i. Equations 1-4 provide the analytical solution to calculated and control length d; or

b. Closed loop where the relative positions of one or more of the following linkage termination points are measured and the length d is adjusted under closed loop control driven by sensor feedback to return the measured parameter to their original position.

i. Point 2 of linkage a's position relative to point 1

ii. Point 3 of linkage c's position relative to point 4

iii. Alternate Point 3 where the patient head meets the head rest

a. The feedback sensors monitoring relative position of the points that define the linkage length could include:

i. Position measurement sensors including but not limited to:
1. Hall effect sensors
2. Magneto-resitive sensors
3. Optical sensors
   a. Encoder
   b. Interferometric
   c. Position sensing detectors

ii. Stress/strain/force/torque monitoring sensors located at the point interfaces that minimize those parameters by adjusting linkage length d under closed loop control.

Referring again to FIGS. 36A-36F, in a preferred exemplary embodiment, the device includes linear actuator linkage d that adjusts the length of the ramp to accommodate the patient, Linkage g that raises the ramp about the Z axis, pivot axis 1, and linkage j that maintains the head support parallel to the X axis by counter rotating about the Z axis along Pivot Axis 4. The device and patient create a 4-bar linkage as defined in Table 1. The associated initial reclined position and relational equations for the angles and actuators d and j as a function of actuators, linkage g length, that drive ramp angle θ_{Z1} are also defined in Table 2 below.

TABLE 2
Patient 4-Bar Linkage & Actuator Linkages

| Initial Conditions when Reclined | | | | | | | | | |
|---|---|---|---|---|---|---|---|---|---|
| X Axis Linkage Pivot Point Definitions | | | | | | | | | |
| | | | | | | Distance from Neck | | | |
| | Reclined (Initial) | | Associated | | Patient | CM to Hip Pivot | | | |
| | Location | | Actuator | Minimum | Adjustment | (cm) | Function | Defining Equations | |
| X Axis Point | Y (cm) | Z (cm) | | | | 59.9 | | | |
| 1 | 0.00 | 0.00 | d | | | | Ramp Pivot Axis | θ_{X1} = θ_{g} − θ_{g0} | θ_{g0} = θ_{g} − θ_{g0} |
| 2 | −11.95 | 6.90 | | | | | Hip Pivot | | |
| 3 | 45.44 | 6.90 | | 6.90 | 0 | | Neck Pivot & Y Neck | | |
| | | | | | | | Adjustment | | |
| 4 | 42.90 | 0.00 | d | 38.4 | 21 | | Head Support Pivot | θ_{X4} = θ_{Z1} | |
| | | | | | | | Axis | | |
| 5 | | | | | | | Head Y Adjustment | | |
| 6 | 9.58 | −3.52 | g | | | | | | |
| 7 | 26.14 | 0.00 | g | | | | | | |
| 8 | 44.00 | −3.40 | j | | | | | | |
| 9 | 65.68 | −1.28 | j | | | | | | |

| Linkage Definitions | | | | | | | | | |
|---|---|---|---|---|---|---|---|---|---|
| | | | | | Linkage/ | Linkage/ | | | |
| | | | | Linkage/ | Opposite | Opposite | | | |
| | First | Second | | Opposite | Angle Value | Angle Value | | | |
| Linkage | Axis | Axis | Length cm | Angle | Rad | Degrees | Actuator? | Function | |
| a | 1 | 2 | 13.80 | θₐ | 2.6180 | 150.00 | | | θₐ = θₐ₀ − θ_{X1} |
| b | 2 | 3 | 59.99 | θ_{b} | 0.0000 | 0.00 | | | θ_{b} = asin [(c sin θ_{c} − |
| | | | | | | | | | a sin θₐ)]/b |
| c | 3 | 4 | 7.35 | θ_{c} | 1.2180 | 69.79 | | | θ_{c} = θ_{c0} + θ_{Z4} |
| d | 1 | 4 | 45.50 | θ_{d} | 0.0000 | 0.00 | 2301002100225G6 | Determines Ramp | d = a cos θₐ + b cos θ_{b} − |
| | | | | | | | Linear Actuator | Length | c cos θ_{c} |
| e | 1 | 6 | 10.21 | θₑ | 0.2094 | 12.00 | | | θₑ = acos[(f² + g² − e²)/2fg] |
| f | 1 | 7 | 26.14 | θ_{f} | 2.5800 | 147.82 | | | θ_{f} = acos[(e² + g² − e²)/2eg] |
| g | 6 | 7 | 16.93 | θ_{g} | 0.3521 | 20.17 | 2331002102050G6 | Determines Ramp | θ_{g} = acos[(e² + f² − g²)/2ef] |
| | | | | | | | Linear Actuator | Incline | |
| h | 4 | 8 | 3.57 | θₕ | 0.1512 | 8.66 | | | θₕ = acos[(i² + j² − h²)/2ij] |
| i | 4 | 9 | 22.82 | θᵢ | 1.8485 | 105.91 | | | θᵢ = acos[(h² + j² − i²)/2hj] |
| j | 8 | 9 | 21.58 | θⱼ | 1.1419 | 65.43 | 2331002102050G6 | Determines Head | θⱼ₀ = acos[(h² + i² − j²)/2hi] |
| | | | | | | | Linear Actuator | Support Incline | |

| Linkage | | |
|---|---|---|
| Definitions | | |
| Linkage | | |
| a | θₐ₀ = 150° for Small-Large Aviator | a = 0.23 b based on Medium Aviator |
| b | θ_{b0} = 0° for Small-Large Aviator | b = (d₀ + 2.54 cm)/(1 − 0.23 cos (180° − θₐ₀)) |
| c | θ_{c0} = acos(2.54 cm/c) | c = (2.54 cm² + (a sin(180° − θₐ₀))²)^{1/2} = (2.54 cm² + Z²)^{1/2} |
| d | | |
| e | | |
| f | | |
| g | | |
| h | | |

| | | |
|---|---|---|
| i | θⱼ = θⱼ₀ + θ_{Z4} | j = (h² + i² − 2hi cos θⱼ)^{1/2} |
| j | θⱼ = θⱼ₀ + θ_{Z4} | j = (h² + i² − 2hi cos θⱼ)^{1/2} |

An illustration in FIG. 32A-32D for a mid-sized male patient with Neck Pivot distance to Hip Pivot distance of 59.9 cm and hip pivot, axis 2, to ramp pivot, axis 2, is provided. The associated Table 3 provides angles and linkage lengths as a function of driving linkage g from a length of 16.75 cm to 22 cm. A plot of Linkage lengths d and j as well as ramp angle θ_{X1} and head support angle θ_{X4} as a function of linkage length g is provided in FIGS. 36A-36F.

TABLE 3
Linkage length commands for d and j as a function of linkage g

| Linkage g | | | | | Linkage d | | | | | | |
|---|---|---|---|---|---|---|---|---|---|---|---|
| (cm) | θ_{X1} (Degrees) | θ_{X1} (Rad) | θ_{X4} (Degrees) | θ_{X4} (Rad) | (cm) | θₐ (Rad) | θ_{b} (Rad) | θ_{c} (Rad) | θ_{g} (Rad) | Linkage j (cm) | θⱼ (Rad) |
| 16.75 | −1.980799995 | −0.0346 | 1.980799995 | 0.0346 | 45.27 | 2.6526 | 0.0070 | 1.2180 | 0.3175 | 21.70 | 1.1765 |
| 17.00 | 0.727219333 | 0.0127 | −0.727219333 | −0.0127 | 45.50 | 2.6053 | −0.0031 | 1.2053 | 0.3648 | 21.53 | 1.1292 |
| 17.25 | 3.168850885 | 0.0553 | −3.168850885 | −0.0553 | 45.52 | 2.5627 | −0.0134 | 1.1627 | 0.4074 | 21.39 | 1.0866 |
| 17.50 | 5.421111271 | 0.0946 | −5.421111271 | −0.0946 | 45.55 | 2.5234 | −0.0228 | 1.1234 | 0.4467 | 21.26 | 1.0473 |
| 17.75 | 7.530863273 | 0.1314 | −7.530863273 | −0.1314 | 45.59 | 2.4866 | −0.0317 | 1.0866 | 0.4836 | 21.13 | 1.0105 |
| 18.00 | 9.529107906 | 0.1663 | −9.529107906 | −0.1663 | 45.65 | 2.4517 | −0.0400 | 1.0517 | 0.5184 | 21.02 | 0.9756 |
| 18.25 | 11.43760228 | 0.1996 | −11.43760228 | −0.1996 | 45.72 | 2.4184 | −0.0479 | 1.0184 | 0.5517 | 20.92 | 0.9423 |
| 18.50 | 13.27230674 | 0.2316 | −13.27230674 | −0.2316 | 45.79 | 2.3863 | −0.0555 | 0.9864 | 0.5838 | 20.82 | 0.9103 |
| 18.75 | 15.04534012 | 0.2626 | −15.04534012 | −0.2626 | 45.88 | 2.3554 | −0.0627 | 0.9554 | 0.6147 | 20.72 | 0.8793 |
| 19.00 | 16.76616329 | 0.2926 | −16.76616329 | −0.2926 | 45.97 | 2.3254 | −0.0697 | 0.9254 | 0.6447 | 20.63 | 0.8493 |
| 19.25 | 18.44233307 | 0.3219 | −18.44233307 | −0.3219 | 46.07 | 2.2961 | −0.0765 | 0.8961 | 0.6740 | 20.54 | 0.8201 |
| 19.50 | 20.08000305 | 0.3505 | −20.08000305 | −0.3505 | 46.17 | 2.2675 | −0.0830 | 0.8676 | 0.7026 | 20.46 | 0.7915 |
| 19.75 | 21.68426774 | 0.3785 | −21.68426774 | −0.3785 | 46.29 | 2.2395 | −0.0894 | 0.8396 | 0.7306 | 20.38 | 0.7635 |
| 20.00 | 23.25940615 | 0.4060 | −23.25940615 | −0.4060 | 46.40 | 2.2120 | −0.0955 | 0.8121 | 0.7581 | 20.31 | 0.7360 |
| 20.25 | 24.80905851 | 0.4330 | −24.80905851 | −0.4330 | 46.53 | 2.1850 | −0.1015 | 0.7850 | 0.7851 | 20.24 | 0.7089 |
| 20.50 | 26.33635727 | 0.4597 | −26.33635727 | −0.4597 | 46.66 | 2.1583 | −0.1074 | 0.7584 | 0.8118 | 20.17 | 0.6823 |
| 20.72 | 27.66406041 | 0.4828 | −27.66406041 | −0.4828 | 46.78 | 2.1352 | −0.1124 | 0.7352 | 0.8349 | 20.11 | 0.6591 |
| 21.00 | 29.33445565 | 0.5120 | −29.33445565 | −0.5120 | 46.94 | 2.1060 | −0.1186 | 0.7060 | 0.8641 | 20.04 | 0.6300 |
| 21.25 | 30.80976336 | 0.5377 | −30.80976336 | −0.5377 | 47.08 | 2.0803 | −0.1240 | 0.6803 | 0.8899 | 19.98 | 0.6042 |
| 21.50 | 32.27183984 | 0.5632 | −32.27183984 | −0.5632 | 47.24 | 2.0547 | −0.1293 | 0.6548 | 0.9154 | 19.92 | 0.5787 |
| 21.75 | 33.7223866 | 0.5886 | −33.7223866 | −0.5886 | 47.40 | 2.0294 | −0.1345 | 0.6294 | 0.9407 | 19.86 | 0.5534 |
| 22.00 | 35.16294644 | 0.6137 | −35.16294644 | −0.6137 | 47.56 | 2.0043 | −0.1395 | 0.6043 | 0.9658 | 19.81 | 0.5282 |

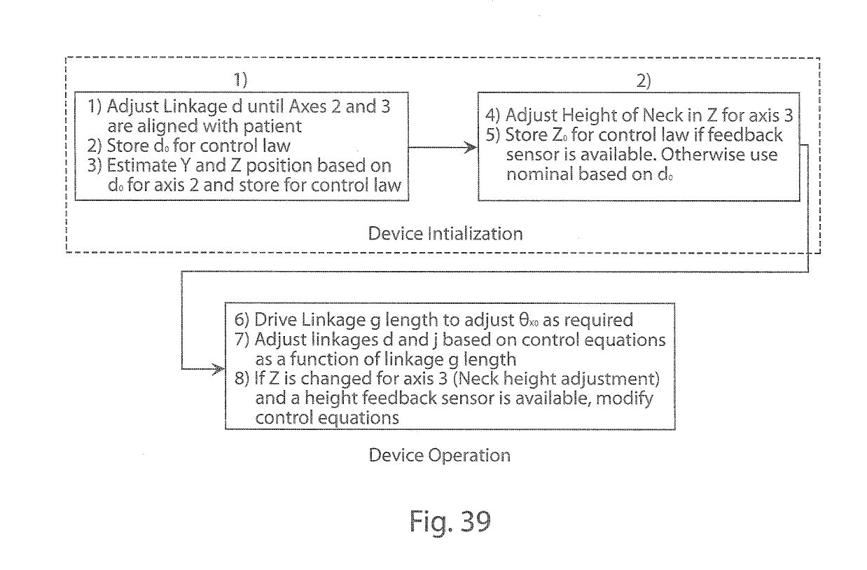

The control steps for the device initialization and operation are outlined in FIG. 39. Optionally, if desired, a sensor may be provided for measuring the Y location of axis 3 which is the neck height adjustment. If a sensor is provided, the actuator control laws that adjust d would be appropriately modified.

An illustration of the jaw thrust parameters and associated Cartesian coordinate system are provided in FIGS. 27-29. In a jaw thrust maneuver, the mandible is moved ΔL_{Thrust}, due to the applied force F_{Thrust}. In order to avoid necrosis of the muscle and epidermal tissue, the maximum allowable pressure that can be applied in a thrust is P_{Thrust}. Given the jaw thrust is applied symmetrically on the left and right side of the mandible, the load on each side is F_{Thrust}/2. Initial requirements for the jaw thrust maneuver are presented in Table 4.

TABLE 4
Jaw Thrust Maneuver Top-Level Requirements

| | Parameter | Value | Comment |
|---|---|---|---|
| 1 | Jaw Thrust | 7 mm-10 mm | The most optimal protrusion of the |
| | Displacement, | @ +22° about | mandible is between |
| | ΔL_{Thrust} | the Y_{Shull} Axis | 7 mm-10 mm, >10 mm does |
| | | | not increase patency²⁴ |
| 2 | Total Jaw | 34.07 ± 9.33 N | The mean value of the highest |
| | Thrust | | forces to accomplish the |
| | Force, F_{Thrust} | | jaw thrust maneuver²⁶ |
| 3 | Maximum | <66 kPa, 4 | Muscle damage occurred at high |
| | allowable | hours | pressure-short duration²⁷ |
| | pressure | | |
| | resulting | | |
| | from Jaw | | |
| | Thrust, P_{Thrust} | | |

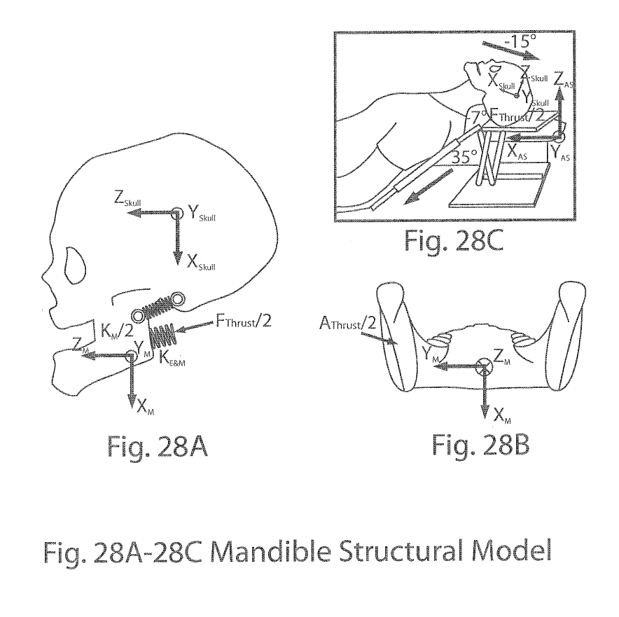

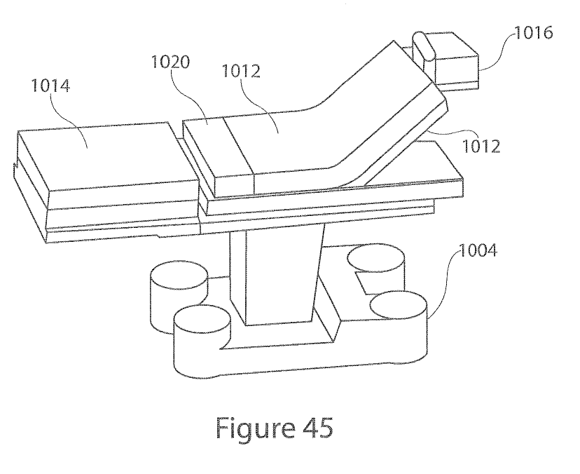

An illustration of a simplified mandible structural model is provided in FIGS. 28A-28C. For the purpose of this illustration, the mandible is assumed to be infinitely stiff with its associated coordinated system X_{M}, Y_{M} & Z_{M}. The back of the skull with its associated coordinate system is tied to ground, with the assumption that it is fixed both in torsion and translation, and the mandible is constrained in all but the thrust axis. Compliance in the mandible is represented by the left and right lateral pterygoid muscle spring stiffness, K_{M}/2, and the effective spring stiffness of the muscle and epidermal tissue below the thrust force, F_{Thrust}/2. Note that when the patient is located on the device, the thrust force vector is applied at a nominal 70 angle about the Y_{AS} axis as illustrated in the top right portion of FIG. 45A. The jaw thrust force, F_{Thrust}, is transmitted over the area, A_{Thrust}, through the muscle/epidermal tissue spring on each side of the mandible, K_{E&M}. A_{Thrust} is determined by the necrosis pressure limit, P_{Thrust}, and time period that the thrust is applied. See Table 5 below.

TABLE 5
Biomechanically Accurate Mandible Model Requirements

| | Parameter | Value | Comment |
|---|---|---|---|
| 1 | Mandible muscle | 4.86 N/mm | Based on Median thrust force of |
| | spring stiffness, K_{M} | | 34 N and extension of 7 mm^{24,26} |
| 2 | Minimum thrust | >6.6 cm2 | Area required with thrust force |
| | area, A_{Thrust} | | of 43.4 N and maximum |
| | | | pressure level allowed, |
| | | | P_{Thrust} of <66 kPa, 4 hours²⁷ |
| 3 | Muscle/epidermal | 11 N/mm | Based on Young's Modulus of |
| | tissue spring on | | 101.20 kPa, 3 mm thickness and |
| | each side of the | | A_{Thrust}/2 Area²⁸ |
| | mandible, K_{E&M} | | |

The patient is positioned on the device with the neck flexion angle at 350 and the plane of the face is −15° to the horizontal. As a result the coordinate system of the skull is rotated about the Y_{Skull} axis and the device y axis, Y_{AS}, by −15° as shown in FIG. 28C. As a result, the nominal jaw thrust force vector is applied at a 7° angle relative to the Y_{AS} axis. Note the thrust angle is 22° relative to the Y_{Skull} axis.

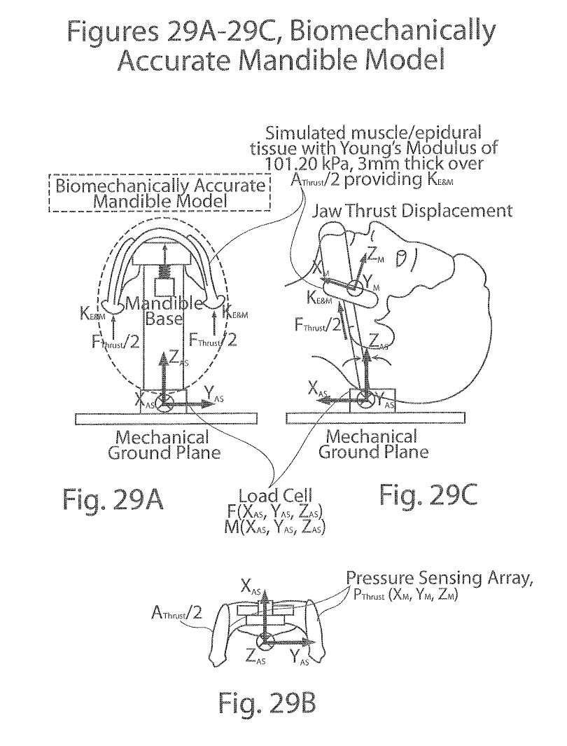

Referring again to FIGS. 29A-29C, a mandible base is connected to structural ground, by a six-degree-of-freedom (DOF) load cell measuring reaction forces and moments. The mandible slides along the base, constrained in all but the ΔL*_{Thrust} axis as shown (70 about the Y_{AS} axis). Stiffness of the left and right lateral pterygoid muscle that provides the thrust resistance, is represented by spring stiffness K_{M}. The simulated mandible consists of an “infinitely” stiff mandible bone covered by an elastomeric material shown in green (silicone rubber is the initial choice due to its similarity in mechanical properties to the skin³⁰). With an elastic modulus of 100 kPa, the effective spring stiffness when a pressure is applied over the area A_{Thrust}/2 with a thickness of 3 mm is K_{E&M} (Epidermis and muscle). Jaw thrust forces are applied to the left and right side of the mandible as shown, or in shear along the sides of the mandible. A pressure sensing grid will be applied to the outer surface of the mandible in order to measure the pressure field when a jaw thrust is applied to the mandible model. Displacement of the mandible along the mandible base will be measured with a dial indicator or other distance measuring device. Details of the load cell and pressure measurement array are provided below.

Multiple jaw thrust approaches can be employed. Two approaches include the jaw thrust approach shown in FIGS. 28A-28C where a thrust moment, M_{Thrust}, applied about the Y_{AS} axis results in a thrust force, F_{Thrust}/2 on the left and right side of the mandible. In this approach the hands-free jaw thrust device is detached from the device base, and jaw cups that interface with the mandible, are shown in FIGS. 29A-29C. A second approach interfaces to the side of the mandible through suction cups providing a shear thrust force. The benefit of a suction cup approach is that there is a larger area to interface on the side, as opposed to the back of the mandible, resulting in lower pressure being required to apply the thrust and reduce the possibility of skin or muscle damage. There is precedence in using vacuum to reduce bruising or other damage, as is the case in vacuum assisted delivery during pregnancy. In all cases, thrust force, due to an applied force or moment and mandible displacement will be measured and the thrust provided under closed loop control of the jaw thrust device.

The jaw thrust force provided by the device will be provided under closed loop feedback measuring P_{Thrust} directly or indirectly, as measured either by pressure array sensors located in the Jaw Cup/Suction Cup-Mandible interface, similar to this used in the mandible model, or in a load cell measuring the applied force F_{Thrust} or moment M_{Thrust}. This feedback will determine the applied thrust force required for the jaw thrust maneuver.

Testing of the Mandible Model with the hands-free jaw thrust device of the present invention involved thrusting the mandible incremental distances of 5 mm-15 mm (In 1 mm steps) at angles about Y_{AS} of 5°-10°. (In 10 steps). Four parameters were be simultaneously measured and recorded as a function of time during testing as illustrated in FIGS. 29A-29C. These include:
1. Thrust pressure field at the mandible interface as measured in the Mandible reference frame, P_{Thrust} (X_{M}, Y_{M}, Z_{M}).
2. Reaction forces and moments as transferred through the mandible, to the skull, to the device mechanical ground plane, F(X_{AS}, Y_{AS}, Z_{AS}) and M (X_{AS}, Y_{AS}, Z_{AS}).
3. Displacement of the mandible relative to the device reference frame, ΔL.
4. Jaw thrust maneuver force, F_{Thrust}, applied by the hands-free jaw thrust device to the mandible, as measured at the hands-free jaw thrust device, F(X_{JC}, Y_{JC}, Z_{JC}) and M (X_{JC}, Y_{JC}, Z_{JC}). Note these are assumed to be the similar to 2.
   Table 6 provides the measurement parameters, requirements and measurement approach for testing. The last column identifies sensor solutions and provides a basis for the requirement.

TABLE 6
Test Measurement Parameters, Requirements & Approach

| | Parameter | Requirement | Candidate Sensor/Basis of Requirement |
|---|---|---|---|
| 1 | Pressure Field Sensor, | | Reference Table, TekScan 4205 is a |
| | P_{Thrust} (X_{M}, Y_{M}, Z_{M}) @ | | candidate sensor |
| | Mandible Interface | | http://www.tekscan.com/4205-pressure- |
| | | | sensor |
| 1.1 | Range | 0-100 kPa | <66 kPa, 4 hours is the preliminary |
| | | | requirement²⁷ |
| 1.2 | Pressure Measurement | 0.7 kPa | Place holder, 1/100 of preliminary |
| | Accuracy | | requirement |
| 1.3 | Pressure Measurement | 0.5 kPa | Place holder, 1/100 of range |
| | Precision | | |
| 1.4 | Pressure Measurement | 1.5 × 10⁻³ m²/ | Approximate area for rear and side of |
| | Sensor Area | 6.0 × 10⁻³ m² | right and left of mandible |
| 1.5 | Pressure Grid spatial | <2 mm in X & Y | Placeholder |
| | resolution | | |
| 1.6 | Pressure Array Minimum | “TBD” | |
| | Surface Radius | | |
| 1.7 | Pressure Measurement | >10 Hz | Placeholder |
| | Bandwidth | | |
| 2 & 4 | Reaction Force/Moment | | Reference Table, Candidate SRI 3702A |
| | Sensor, F (X_{AS}, Y_{AS}, Z_{AS}) | | is a candidate sensor, |
| | and M (X_{AS}, Y_{AS}, Z_{AS}) | | http://www.srisensor.com/pdf/m3202.pdf |
| 2.1 | Force Range | −50 N-+50 N | 34.07 ± 9.33 N is expected jaw thrust |
| | | | force²⁶ |
| 2.2 | Moment Range | −0.75 N-m to | Placeholder |
| | | +0.75 N-m | |
| 2.3 | Force Accuracy | <1 N, 1 σ | Placeholder |
| 2.4 | Force Precision | 0.05 N, 1 σ | Placeholder |
| 2.5 | Measurement Bandwidth | >10 Hz | Placeholder |
| 3 | Mandible Displacement | | Reference Table, Candidate sensor is |
| | Sensor | | “TBD” |
| 3.1 | Length of Jaw Thrust | 0.7 mm-10.0 mm | Distance required to achieve airway |
| | displacement, θL_{Thrust} | | opening and acceptable glottic view²⁴ |
| 3.2 | Measurement Accuracy | 0.01 mm, 1σ | |
| 3.3 | Measurement Precision | 0.005 mm, 1 σ | Placeholder |
| 3.4 | Measurement Bandwidth | >10 Hz | Placeholder |

The present invention is believed to be the first of its kind to combine several different mechanisms to maximize ventilation in a hands-free fashion. The device is also novel in that it is the only patient positioning device that can adjust head and/or neck position in real time during intubation. This is essential because the sniffing position is not the optimal intubating position for every patient, although it is for most. The invention being described has the ability to accommodate for those patients whose glottis is not in view in the sniffing position by being able to adjust the head and/or the neck until the glottis is in view.

The device is also novel because it is the only patient positioning device that secures a patient's head and neck in optimal ventilating and intubating position and can provide nasal continuous positive pressure ventilation while simultaneously attempting intubation. This helps significantly prolong the short yet critical time period between the patient's last breath and securing of the endotracheal tube.

The present invention also has an ability to perform a jaw thrust maneuver in the lateral position, along with the ability to displace weight off of a patient's chest by utilizing a lateral decubitus position, along with placing the patient in the sniffing position which aligns all 3 axes (oropharyngeal, laryngeal, tracheal). The invention also gives the provider the ability to first try a much lesser invasive way of maintaining a patent airway by placing the patient in the proper sniffing position without eliciting any painful stimuli. The provider can then restrain the patient's head to prevent the patient from being displaced from the desired position. If the patient's airway is not yet patent, the provider can adjust the mandibular arms with ratchet capability, each applied to each side of the mandible to displace the jaw forward. If the patient still cannot maintain a patent airway, the present invention, by placing the patient in the desired position, aligns all 3 axes in order to achieve the best possible view to intubate the patient.

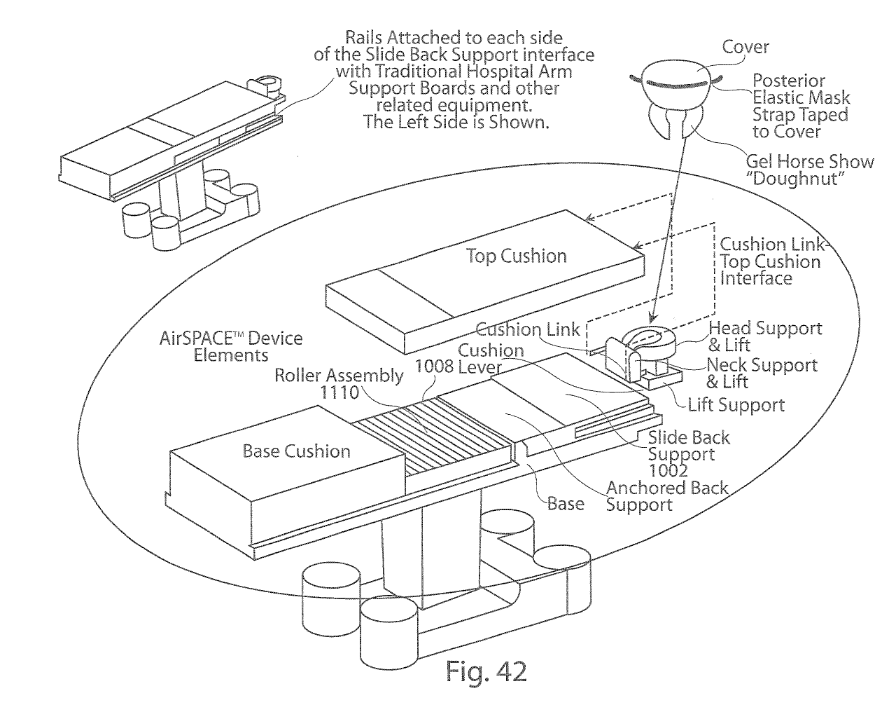

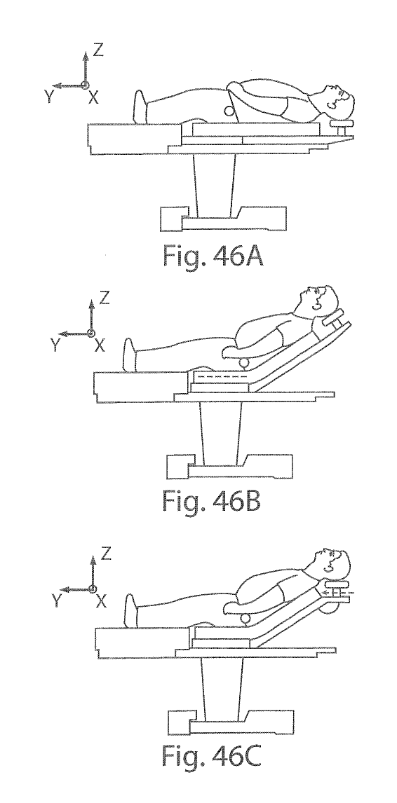

FIGS. 42-46C illustrate another and preferred embodiment of the positioning device 1002 of the present invention installed on a conventional operating table 1004. More particularly, there is shown an alternate method of maintaining the patient's head and head support, linkages axis 3, in a collocated position as the device is raised or lowered or as the lift support is raised and lowered is to allow the hip axis 2 (X₂) to be allowed to translate along the Y and Z axis as illustrated in FIGS. 46A-46C. The alternate method consists of connecting the top cushion on which the patient is lying to the slide back support and allowing the top cushion to slide relative to the anchored back support if that length is adjusted and on the roller assembly along the Y axis. Additionally, as the lift support rotates about the X axis, the head, on the head support slides along the Y axis to compensate for the change in angle and linkage length between axis 3 and 4.

FIG. 46A shows the patient lying in a supine position with the head and head support collocated at linkage axis 3. As the ramp is raised to an incline position by rotating about the X₁ axis, the patient on the top cushion both slide on the device along the Y axis, FIG. 46B. The head and head support remain collocated and the hip axis 2 is allowed to move.

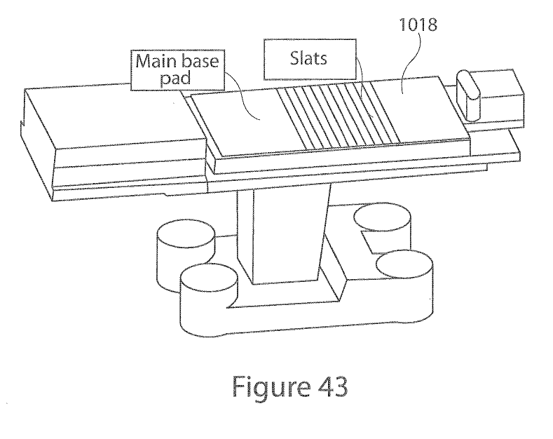

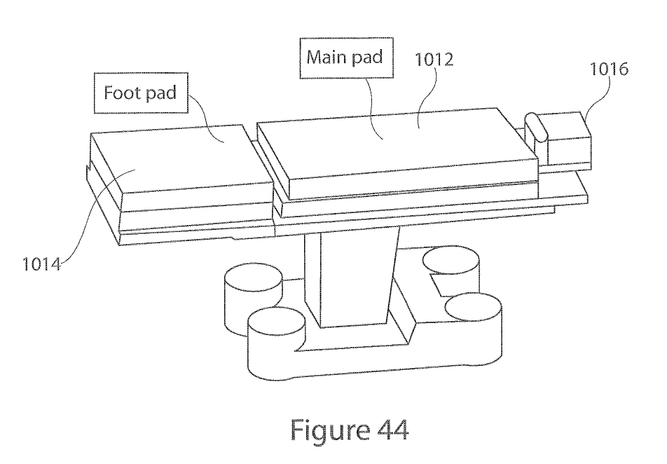

Referring first to FIG. 42, a foot spacer 1006 and a base spacer 1008, each having a thickness equal to the thickness of the positioning device 1002, are placed on the operating table so as to provide a level surface. Foot spacer 1006 is formed, for example, of a high density foam. Base spacer 1008 preferably includes a plurality of rollers 1010 to permit the main pad 1012 (FIG. 44) to roll back and forth as the positioning ramp is raised and lowered or extended as previously described. A foot pad 1014 is also provided, and main paid 1012 and foot pad 1014 are of the same thickness so as to approximate the height of the head and neck support 1016 of the positioning device. Referring also to FIG. 43, the main paid 1012 includes a slatted or pleated base pad 1018 which allow the support structure to bend to support the elevation of the ramp over the range of the ramp extension. The assembled unit is shown in FIG. 45. Also, if desired, additional spacer pads 1020 may be inserted between the foot pad 1014 and the main paid 1012 when the positioning device 1002 is extended, to support taller patients. See also FIGS. 46A-46C.

While the invention has been described in detail with reference to exemplary embodiments thereof, various changes can be made, and equivalents employed, without departing from the scope of the invention. By way of example, the nasal mask, oral mask, and/or full facemask can be used for nebulizer treatments. Also, the nasal mask, oral mask, and full facemask can be used to measure End-Tidal CO2 (EtCO2) or capnography. Additionally, the nasal mask, oral mask, and full facemask also consists of patent tubing which consists of two ends to be used as an gas source to transport patients, where the distal end of the tubing is connected to either a stand alone or a portable generator for the supply of gas at a pressure below, equal to, or elevated above atmospheric pressure; a gas delivery conduit coupled to said generator a portable gas supply (oxygen, anesthetic gases, air, or any other gases) and the proximal end is connected to an adaptor, which contains an End-Tidal CO2 port, a nebulizer port, a PEEP valve port, expiratory port and/or valve, pressure relief valve, which has an aperture which attaches to either the nasal mask, the oral mask, or the full face mask.

In yet other embodiments the nasal mask, oral mask, and/or full face mask can be connected to a generator for the supply of gas, where the amount and concentration of gas delivered is controlled by the supply source as well as the expiratory port.

In yet other embodiments the nasal mask, oral mask, and/or full face mask can be used as a scavenger system by connecting the nasal mask and the oral mask simultaneously, where the nasal mask can be used to deliver positive pressure and the oral mask can be connected to a suctioning device to properly store and/or dispose gases.

A feature and advantage of the present invention is that the nasal mask will contour around the nasal bridge, nose, and upper lip in such a way that it and the generator gas supply it is connected to does not interfere with the operator's access to the mouth/oral cavity, lips, cheeks, chin, jaw, and neck.

Also, the nasal mask and full face mask can be connected to a resuscitator bag with or without a gas supply attached to the resuscitator bag. Still other modifications are possible. Still other features and advantages of the present invention include:
Neck and head flexion for optimizing the view as well as achieving the Sniff position can be accomplished with one or some combination of the following:
   a. Neck Support Lift translation along the Z axis
   b. Head Support Lift translation along the Z axis
   c. Lift Support rotation about Linkage Axis 4 (X axis) (The head support lift and neck support lift are attached to the Lift Support)
   d. Note one embodiment may not have the Neck Support Lift
The mask anchor anterior strap may have 2 or more straps with one strap securing the chin.
The mask anchor straps attach to the top surface of the head support lift, that also contains a soft gel-like doughnut to support the head, with friction wedge.
The top of the head support lift with a soft doughnut-like gel that supports the head is detachable from the Lift base. This allows the top of the head support lift with the gel doughnut and mask anchor to be secured to the patient if there is the need to move the patient to a different part of the OR table without the patient positioning device.
The top of the head support lift is covered with a disposable barrier. The top of this barrier has an elastic strap taped to it that can be removed and posteriorly attach to the mask that also has an oxygen port. The ventilation port serves as a CO2 exhaust port when the oxygen line is attached to the oxygen port that supplies O2 to the patient. This allows the mask to also be used as an oxygen supply mask postoperatively.
The mask anchor connection that holds the anterior mask straps as well as the posterior elastic strap wedge can be integrally part of the mask, as opposed to a separate component that was shown in other embodiments.

The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

1: A nasal mask which can be used separately, or with an oral mask releasibly attached to the nasal mask through a closable opening on the nasal mask.

2: The nasal mask as claimed in claim 1, wherein when the oral mask is engaged with the nasal mask, the closable opening allows gases to flow bilaterally between the oral mask and the nasal mask.

3: The nasal mask as claimed in claim 1, wherein when the oral mask is combined with and engaged with the nasal mask, the combination creates a face mask which can be used for bag-mask ventilation, general anesthesia, deep sedation or respiratory treatment.

4: The nasal mask as claimed in claim 1, wherein when nasal mask is used by itself, the closable opening automatically closes whereupon the nasal mask may be used for nasal Bipap/CPAP, ventilation during intubation, general anesthesia, deep sedation, respiratory treatment, delivery of oxygen, or treatment of obstructed sleep apnea.

5: The nasal mask of claim 1, wherein the closable opening comprises a door, and the oral mask includes a connector which when engaged with the nasal mask door, pushes the door open.

6: A nasal mask, or combined nasal mask/oral mask full face mask, comprising:
a nasal mask body adapted for connection with a supply of breathable gas, preferably oxygen, air, or air anesthetic gas;
a cushion secured to said nasal mask body, the body and cushion forming a nose-receiving cavity, said cushion including: a nasal bridge region, a cheek region and an upper lip region;
a substantially triangularly-shaped first nasal membrane formed of resilient material having a first molded inwardly curved rim to surround patient's nose;
a second nasal membrane formed of resilient material, said second membrane being relatively more flexible than said first nasal membrane, said second nasal membrane having a second molded inwardly curved rim, said second molded rim being of the same general shape as said first molded rim and fixed to and extending away from said first nasal membrane so as to provide a second nasal membrane inner surface spaced a first distance from an outer surface of said first molded rim in said cheek region and said second membrane inner surface spaced a second distance from said first nasal membrane outer surface of said first molded rim in said nasal bridge region, said second distance greater than said first distance, said first and second distances measured when the mask is not in use, a portion of said second molded rim forming a face contacting seal; and
wherein said seal portion is substantially coterminous with respect to said second molded rim and is resiliently deformable towards said first nasal membrane when said mask is in use.

7: The mask as claimed in claim 6 further comprising tubing for connection to a generator for the supply of gas at a pressure below, equal to, or elevated above atmospheric pressure;
a gas delivery conduit having one end for coupling to said supply of gas, and its other end for coupling to an adaptor, which contains an End-Tidal CO₂ port, a nebulizer port, a PEEP valve port, expiratory port and/or valve, pressure relief valve, which has an aperture which attaches to either the nasal mask, the oral mask, or the full face mask.

8: The mask as claimed in claim 6, wherein the mask is connectable to a generator for the supply of gas, wherein an amount and concentration of gas delivered is controlled by a supply source as well as the expiratory port, and/or used as a scavenger system by connecting the nasal mask and the oral mask simultaneously, where the nasal mask can be used to deliver positive pressure and the oral mask can be connected to a suctioning device to properly store and/or dispose gases.

9: The mask as claimed in claim 6, wherein the nasal mask is contoured for the patient's nasal bridge, nose, and upper lip such that when the mask and the generator gas supply are connected and worn by a patient, the mask does not interfere with the operator's access to the mouth/oral cavity, lips, cheeks, chin, jaw, and neck of the patient, and/or connected to a resuscitator bag with or without a gas supply attached to the resuscitator bag.

10: The mask as claimed in claim 6, wherein said nasal mask body includes either integrated head strap attachment points using either an anterior approach or posterior approach or separate head strap attachment points using either an anterior approach or a posterior approach when placed over the mask, which mask when combined with straps attached to a surface secure the mask to the patient's face to ensure a tight seal and to maintain the patient's head in a desired position to maintain airway patentcy.

11: The mask as claimed in claim 6 further comprising securing straps fixed to said attachment points on the mask.

12: The mask as claimed in claim 6, wherein said second nasal membrane molded rim and said first nasal membrane molded rim each have co-located notches to accommodate a bridge of a nose of a wearer.

13: The mask as claimed in claim 6, wherein said first and second molded rims are substantially saddle-shaped.

14: The mask as claimed in claim 6, wherein said second nasal membrane is shaped so that said seal portion, in use, contacts at least a bridge of a nose of a wearer.

15: The mask as claimed in claim 6, wherein said seal portion, in use, contacts facial tissue around the sides and over a bridge of the nose of the wearer, and between the base of the wearer's nose and an upper lip of the wearer.

16: The mask as claimed in claim 6, wherein said rim and said seal portion are shaped to generally match facial contours in the region of facial tissue around sides and over a bridge of a nose of the wearer, and between a base of the nose and an upper lip of the wearer.

17: A nasal mask for connection to a patient's face comprising:
a mask body for connection with a supply of breathable gas; and a mouth cushion secured to said mask body, the mask body and mouth cushion forming a mouth-receiving cavity, said mouth cushion including: a mouth region, a cheek region and an upper and lower lip region;
a substantially oval-shaped first mouth membrane of resilient material having a first molded inwardly curved rim to surround a patient's nose;
a second mouth membrane also of resilient material, said second mouth membrane being relatively more flexible than said first mouth membrane, said second mouth membrane having a second molded inwardly curved rim, said second molded rim being of the same general shape as said first molded rim and fixed to and extending away from said first mouth membrane so as to have a second mouth membrane inner surface spaced a first distance from an outer surface of said first molded rim in said cheek region and said second mouth membrane inner surface spaced a second distance from said first mouth membrane outer surface of said first molded rim in said mouth region, said second distance greater than said first distance, said first and second distances measured when the mask is not in use, a portion of said second molded rim forming a face contacting seal;
wherein said seal portion is substantially coterminous with respect to said second molded rim and is resiliently deformable towards said first mouth membrane when said mask is in use.
